(19) United States
(12) Patent Application Publication
(10) Pub. No.: US 2026/0301872 A1
(43) Pub. Date: Oct. 1, 2026

(54) METHOD, SYSTEM, AND COMPUTER-READABLE MEDIA FOR OPTIMIZING BIOLOGICAL INFORMATION STORAGE AND PROCESSING USING RECOGNITION SCIENCE DERIVED PRINCIPLES

(71) Applicant: Jonathan Washburn, Austin, TX (US)

(72) Inventor: Jonathan Washburn, Austin, TX (US)

(21) Appl. No.: 19/629,558

(22) Filed: Mar. 26, 2026

Related U.S. Application Data
(60) Provisional application No. 63/779,010, filed on Mar. 27, 2025.

(51) Int. Cl. G16B 35/10; G06F 30/20

(52) U.S. Cl. CPC G16B 35/10; G06F 30/20

(57) ABSTRACT

A computer-implemented method, system, and non-transitory computer-readable medium are disclosed for designing biological information-bearing polymers using recognition science derived metrics. Input data defining a target biological application and one or more candidate polymers or sequence spaces is received. For at least one candidate, a structural integrity metric, a melting or separation resistance metric, a binding specificity metric, and a replication or transcription fidelity metric are determined. A selection result is then determined based at least in part on the metrics, and one or more selected biological information-bearing polymers are output for physical synthesis, assembly, storage, screening, or use. In some embodiments, the method is used for nucleic acid data storage, aptamer design, probe design, biosensor design, coding-sequence optimization, regulatory-sequence optimization, guide-sequence design, donor-template design, RNA design, or genome-editing-related design.

### CROSS-REFERENCE TO RELATED APPLICATIONS

This application claims the benefit of U.S. Provisional Patent Application No. 63/779,010, filed on Mar. 27, 2025, under 35 U.S.C. 119(e), the entire disclosure of which is hereby incorporated by reference herein in its entirety.

### BACKGROUND

The present invention relates generally to computational biology, molecular genetics, bioinformatics, synthetic biology, nucleic acid engineering, biological information storage, and biological information processing, and more particularly to methods, systems, and computer-readable media for designing, evaluating, optimizing, and selecting biological information-bearing polymers, including DNA, RNA, DNA-RNA hybrids, modified nucleic acids, and related constructs, using recognition science derived principles.

Biological systems store, transmit, interpret, and process information through molecular structures whose performance depends not only on primary sequence composition, but also on higher-order structural, energetic, contextual, and interaction-dependent properties. Nucleic acid molecules, including DNA and RNA, are used in nature and in engineered systems as storage media, templates, guides, regulatory elements, recognition partners, coding sequences, probes, aptamers, and molecular control components. In each of these applications, practical performance is affected by multiple interrelated factors including persistence of structure, resistance to disruption, recognition selectivity, copying accuracy, expression behavior, off-target interaction behavior, and compatibility with biological or synthetic processing environments.

Existing design approaches tend to address these properties in fragmented ways. For example, sequence stability is often estimated using empirical nearest-neighbor thermodynamic parameters fit to experimental melting measurements. Binding performance is often assessed through screening pipelines, motif heuristics, or experimentally intensive enrichment procedures. Replication and transcription fidelity are often treated as separate biochemical subjects, analyzed using polymerase-specific measurements without a unified sequence-design framework. Regulatory sequence design, aptamer design, coding-sequence design, guide-RNA design, and nucleic acid data storage design are likewise often treated as distinct optimization problems with different scoring systems and different assumptions.

As a result, conventional tools frequently optimize only a narrow subset of relevant characteristics. A sequence that appears favorable under one scoring method may perform poorly under another relevant criterion. For example, a sequence chosen for local binding behavior may exhibit poor persistence, poor synthesis performance, increased off-target susceptibility, poor copying fidelity, or unfavorable secondary structure. Likewise, a sequence selected for data density or coding efficiency may be suboptimal with respect to structural persistence, degradation resistance, polymerase compatibility, or recognition selectivity.

Conventional methods also frequently rely on purely empirical scoring schemes that are difficult to generalize across use cases, environmental conditions, or sequence classes. Many such methods are application-specific and do not provide a common framework for jointly evaluating stability, specificity, fidelity, and other performance characteristics in a unified manner. In many settings, this leads to iterative trial-and-error design, increased experimental burden, and incomplete exploration of the available sequence space.

There remains a need for a unified computational design framework capable of evaluating biological information-bearing polymers across multiple dimensions of performance. There is a further need for a design framework that can represent candidate sequences or constructs in a way that captures sequence properties together with structural, energetic, and contextual information, and that can use such information to rank, optimize, and select candidates for practical biological use.

There further remains a need for a method that can support multiple biological applications within a common architecture, including nucleic acid data storage, high-fidelity gene synthesis, aptamer design, biosensor design, transcription-factor binding-site design, coding-sequence optimization, regulatory sequence optimization, genome-editing guide design, RNA therapeutic design, genome stabilization applications, and related uses.

There further remains a need for computational tools capable of evaluating candidate biological sequences using metrics that reflect not only local base composition or nearest-neighbor energetics, but also broader structural integrity, resistance to separation or degradation, target recognition specificity, copying fidelity, and related recognition-dependent properties.

There further remains a need for systems and methods that can generate candidate biological sequences, evaluate such candidates using multiple metrics, compare or rank the candidates under one or more optimization objectives, and output one or more optimized sequences, libraries, constructs, or synthesis-ready designs for physical implementation.

### BRIEF SUMMARY OF THE INVENTION

In some embodiments, the present invention provides a computer-implemented method for optimizing biological information storage and processing by evaluating candidate biological information-bearing polymers using one or more recognition science derived metrics. The method may include receiving one or more candidate biological sequences or candidate sequence spaces, receiving one or more design goals or application constraints, determining one or more metrics for each candidate, and selecting, ranking, or outputting one or more candidates based on the determined metrics.

In some embodiments, the candidate biological information-bearing polymer comprises DNA. In some embodiments, the candidate biological information-bearing polymer comprises RNA. In some embodiments, the candidate biological information-bearing polymer comprises a DNA-RNA hybrid, a modified nucleic acid, a coding sequence, a regulatory sequence, a guide sequence, a probe, an aptamer, a storage oligonucleotide, a donor template, a vector sequence, or a portion thereof.

In some embodiments, a candidate biological construct is represented using one or more descriptors including a sequence descriptor S, a structural descriptor H, and an energetic descriptor E. In some embodiments, one or more environmental, contextual, target-specific, or constraint-specific descriptors are additionally included. In some embodiments, the candidate biological construct is represented as D=(S, H, E). In other embodiments, the candidate biological construct is represented using a broader descriptor set that further includes one or more environmental or application-specific parameters.

In some embodiments, the one or more metrics include a structural integrity metric, a melting or separation resistance metric, a binding specificity metric, and a replication or transcription fidelity metric. In some embodiments, one or more additional metrics are used, including manufacturability metrics, off-target penalty metrics, accessibility metrics, degradation resistance metrics, repair compatibility metrics, immunogenicity-related metrics, expression-related metrics, secondary structure metrics, or combinations thereof.

In some embodiments, the structural integrity metric is configured to evaluate a degree to which a candidate sequence preserves a desired structural or conformational state over a length, region, or coordinate domain. In some embodiments, the structural integrity metric is based on a deviation of a structural profile from a reference or idealized profile.

In some embodiments, the melting or separation resistance metric is configured to evaluate resistance of a candidate sequence to denaturation, dissociation, separation, or related loss of stored or processable biological information. In some embodiments, the melting or separation resistance metric is represented by a function of sequence stability and energetic coherence.

In some embodiments, the binding specificity metric is configured to evaluate selective recognition of a desired target relative to one or more undesired targets. In some embodiments, the binding specificity metric is represented by a ratio of on-target interaction magnitude to off-target interaction magnitude.

In some embodiments, the replication or transcription fidelity metric is configured to evaluate a relative preference for correct copying, interpretation, or processing events as compared to incorrect events. In some embodiments, the fidelity metric is based on a comparison between a correct-event compatibility quantity and an incorrect-event compatibility quantity.

In some embodiments, one or more recognition science derived functions are used in determining one or more of the foregoing metrics. In some embodiments, candidate sequences are ranked using a composite objective function based on weighted combinations of the one or more metrics.

In some embodiments, the method includes generating candidate sequences by one or more of enumeration, mutation, substitution, codon optimization, motif-preserving editing, stochastic search, evolutionary search, machine-learning guided search, assay-feedback guided search, or combinations thereof. In some embodiments, hard constraints and soft constraints are imposed on candidate generation or candidate acceptance.

In some embodiments, the method outputs one or more optimized biological sequences, one or more ranked candidate sets, one or more sequence libraries, one or more synthesis-ready constructs, one or more manufacturing instructions, one or more assay plans, one or more database records, or combinations thereof.

In some embodiments, the invention provides a system comprising one or more processors and one or more non-transitory computer-readable media storing instructions that, when executed by the one or more processors, cause the system to perform any of the methods described herein.

In some embodiments, the invention provides a non-transitory computer-readable medium storing instructions that, when executed by one or more processors, cause the one or more processors to receive candidate biological sequences, determine one or more recognition science derived metrics for the candidate biological sequences, rank or select one or more candidate biological sequences based at least in part on the one or more recognition science derived metrics, and output one or more optimized biological sequences or constructs.

In some embodiments, the invention provides an optimized biological information-bearing polymer produced or selected according to any of the methods described herein. In some embodiments, the optimized biological information-bearing polymer is synthesized, cloned, amplified, transcribed, packaged, stored, screened, expressed, delivered, or otherwise physically implemented after selection.

In some embodiments, the disclosed methods, systems, and media are used in connection with nucleic acid data storage, aptamer design, biosensor design, coding-sequence optimization, regulatory sequence optimization, guide-RNA design, donor-template design, RNA therapeutic design, genome stabilization, sequence library generation, or related applications.

### BRIEF DESCRIPTION OF THE DRAWINGS

The accompanying drawings are included to provide a further understanding of the disclosed embodiments and are incorporated in and constitute a part of this specification. The drawings illustrate representative embodiments of the invention and, together with the description, serve to explain principles of the invention.

FIG. 1 is a schematic diagram of an example system for optimizing biological information storage and processing, the system including an input interface, a candidate generation engine, a metric computation engine, a scoring and ranking engine, and an output interface.

FIG. 2 is a flow diagram of an example method for optimizing a biological information-bearing polymer, including receiving candidate sequences or sequence spaces, determining one or more optimization metrics, ranking or selecting one or more candidates, and outputting one or more optimized biological sequences or constructs.

FIG. 3 is a schematic diagram illustrating an example descriptor framework for representing a candidate biological construct using a sequence descriptor, a structural descriptor, and an energetic descriptor, and optionally one or more environmental, contextual, target-specific, or constraint-specific descriptors.

FIG. 4 is a schematic diagram illustrating an example metric determination framework for evaluating a candidate biological sequence using a structural integrity metric, a melting or separation resistance metric, a binding specificity metric, and a replication or transcription fidelity metric.

FIG. 5 is a schematic diagram illustrating an example composite scoring and candidate selection process, including weighted combination of multiple metrics, normalization or transformation of metric values, thresholding, ranking, and selection of one or more optimized candidates.

FIG. 6 is a schematic diagram of an example computing environment for implementing the disclosed methods, the computing environment including one or more processors, memory, storage, software modules, and one or more user or network interfaces.

FIG. 7 is a schematic diagram illustrating an example embodiment directed to nucleic acid data storage, aptamer design, biosensor design, coding-sequence optimization, regulatory sequence optimization, or related biological sequence-design applications using the disclosed optimization framework.

FIG. 8 is a schematic diagram illustrating an example validation and output workflow, including generation of optimized sequences, ranked candidate libraries, synthesis-ready constructs, assay plans, measured-result feedback, and database records.

### DETAILED DESCRIPTION OF THE INVENTION

Reference will now be made in detail to exemplary embodiments of the invention, examples of which are illustrated in the accompanying drawings. Wherever possible, the same reference numerals will be used throughout the drawings and the following description to refer to the same or like parts.

In general, the invention provides methods, systems, and computer-readable media for optimizing biological information storage and processing by evaluating candidate biological information-bearing polymers according to one or more recognition science derived metrics and selecting, ranking, or outputting one or more candidates for practical use. In preferred embodiments, the biological information-bearing polymer comprises a nucleic acid sequence, including DNA, RNA, a DNA-RNA hybrid, or a modified nucleic acid. In preferred embodiments, the disclosed framework is implemented as a computer-implemented design and optimization platform that receives candidate sequence information, evaluates one or more metric values for each candidate, and outputs one or more optimized sequences, constructs, libraries, or synthesis-ready designs.

The disclosed platform improves computer-based biological sequence design by providing a unified descriptor-and-metric architecture that (i) reduces candidate-space search burden by enabling early rejection via multi-metric thresholds, (ii) improves computational comparability across different biological applications by using a common metric set derived from recognition-science functions, and (iii) produces synthesis-ready outputs tied to physical implementation constraints. These improvements are realized in the operation of the candidate generation, metric computation, and selection modules described herein.

In some embodiments, the invention is used to optimize a sequence for long-term biological information storage, resistance to strand separation, target recognition specificity, replication fidelity, transcription fidelity, or combinations thereof. In some embodiments, the invention is used to optimize storage oligonucleotides, coding sequences, regulatory sequences, probes, aptamers, donor templates, guide sequences, vector sequences, or multi-region constructs that include two or more such regions.

In some embodiments, the invention uses recognition science derived principles to define one or more quantitative measures associated with stability, recognition, compatibility, or information persistence. In preferred embodiments, such measures are used to determine a structural integrity metric, a melting or separation resistance metric, a binding specificity metric, and a replication or transcription fidelity metric. In some embodiments, these metrics are used individually. In other embodiments, two or more of the metrics are combined in a weighted or otherwise transformed objective function.

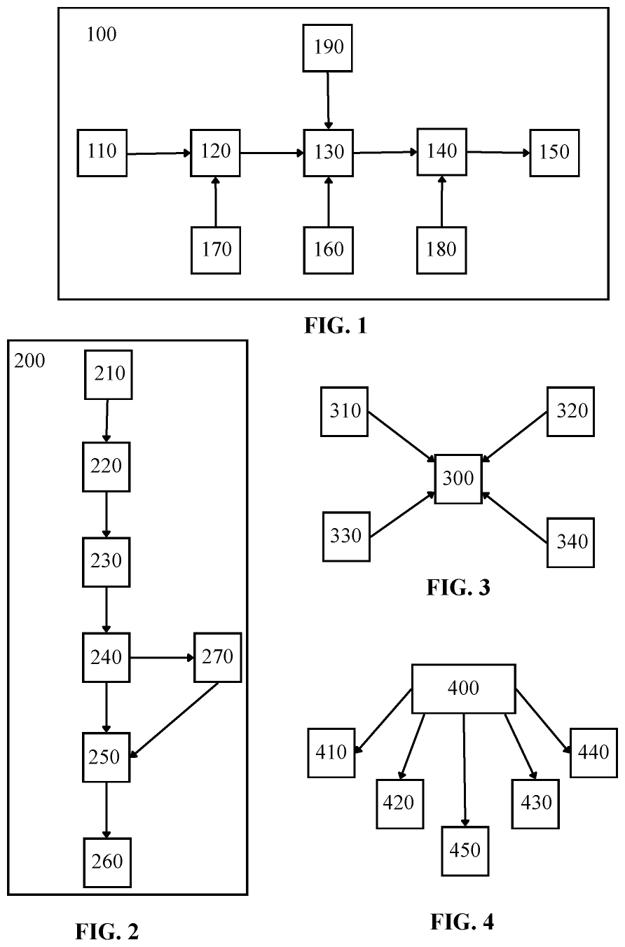

Referring first to FIG. 1, an example system 100 for optimizing biological information storage and processing is shown. In the illustrated embodiment, system 100 includes an input interface 110, a candidate generation engine 120, a metric computation engine 130, a scoring and ranking engine 140, an output interface 150, and a data store 160. In some embodiments, system 100 further includes a constraint engine 170, a calibration or feedback engine 180, and an external resource interface 190.

Input interface 110 is configured to receive one or more candidate biological sequences, sequence templates, sequence libraries, sequence spaces, target definitions, design goals, optimization modes, environmental parameters, assay inputs, or user-defined constraints. In some embodiments, input interface 110 receives an explicit nucleotide sequence. In some embodiments, input interface 110 receives a partially specified sequence with one or more variable positions. In some embodiments, input interface 110 receives a region map identifying coding regions, regulatory regions, binding regions, spacer regions, primer regions, barcode regions, payload regions, flanking regions, or combinations thereof.

Candidate generation engine 120 is configured to generate one or more candidate biological constructs for evaluation. In some embodiments, candidate generation engine 120 generates candidates by enumeration of permitted sequence variants. In some embodiments, candidate generation engine 120 generates candidates by mutation, substitution, synonymous codon replacement, motif-preserving editing, insertion, deletion, recombination, stochastic search, evolutionary search, machine-learning guided search, or combinations thereof. In some embodiments, candidate generation engine 120 operates on an entire construct. In other embodiments, candidate generation engine 120 operates on one or more local regions while preserving one or more fixed regions.

Metric computation engine 130 is configured to determine one or more metric values for each candidate. In preferred embodiments, metric computation engine 130 determines at least a structural integrity metric, a melting or separation resistance metric, a binding specificity metric, and a replication or transcription fidelity metric. In some embodiments, metric computation engine 130 additionally determines one or more secondary structure metrics, manufacturability metrics, degradation resistance metrics, off-target penalty metrics, accessibility metrics, repair compatibility metrics, or expression-related metrics.

Scoring and ranking engine 140 is configured to compare candidate constructs according to one or more optimization objectives. In some embodiments, scoring and ranking engine 140 ranks candidates based on a weighted combination of metric values. In some embodiments, scoring and ranking engine 140 applies one or more normalization functions, threshold filters, dominance rules, or Pareto selection criteria. In some embodiments, scoring and ranking engine 140 outputs a single best candidate. In other embodiments, scoring and ranking engine 140 outputs a ranked candidate list, a filtered library, a Pareto-optimal set, or one or more candidates satisfying one or more threshold conditions.

Output interface 150 is configured to output one or more optimized biological sequences, sequence libraries, constructs, assay plans, manufacturing instructions, database records, synthesis-ready files, or combinations thereof. In some embodiments, output interface 150 outputs a nucleotide sequence in a format suitable for direct synthesis. In some embodiments, output interface 150 outputs a ranked list of candidates together with metric values, weights, predicted performance values, and recommended validation steps.

Data store 160 is configured to store one or more input sequences, candidate sequences, metric values, parameter values, target definitions, off-target definitions, assay results, optimization histories, calibration values, sequence annotations, environmental settings, or output records. In some embodiments, data store 160 stores previously evaluated candidates and allows reuse of computed metric values or partial results. In some embodiments, data store 160 stores regional annotations and region-specific constraints for multi-region constructs.

Constraint engine 170, when present, is configured to enforce one or more hard or soft constraints during candidate generation or candidate acceptance. In some embodiments, the constraints include sequence length constraints, GC-content constraints, forbidden motif constraints, homopolymer constraints, codon preservation constraints, amino-acid preservation constraints, target-region preservation constraints, synthesis constraints, cloning constraints, vector compatibility constraints, or combinations thereof. In some embodiments, one or more hard constraints must be satisfied for a candidate to be accepted for scoring. In some embodiments, one or more soft constraints contribute to a penalty term.

Calibration or feedback engine 180, when present, is configured to receive measured, simulated, inferred, or user-supplied data and to update one or more parameter values, normalization rules, weighting values, or prediction mappings used by metric computation engine 130 or scoring and ranking engine 140. In some embodiments, calibration or feedback engine 180 receives assay data obtained from selected candidates and uses the assay data to refine later candidate evaluations. In some embodiments, calibration or feedback engine 180 supports a closed-loop optimization workflow.

External resource interface 190, when present, is configured to communicate with one or more external databases, synthesis vendors, assay platforms, computational services, laboratory information systems, genomic databases, motif databases, target panels, off-target panels, or combinations thereof. In some embodiments, external resource interface 190 retrieves a target sequence set, a reference genome, a codon usage table, a protein-binding motif database, or a degradation-risk model.

Referring now to FIG. 2, an example method 200 for optimizing a biological information-bearing polymer is shown. In the illustrated embodiment, method 200 includes receiving one or more candidate sequences or sequence spaces at step 210, receiving one or more target definitions or design constraints at step 220, generating or selecting one or more candidate constructs at step 230, determining one or more optimization metrics at step 240, ranking or selecting one or more candidates at step 250, and outputting one or more optimized biological sequences or constructs at step 260.

At step 210, method 200 receives one or more candidate sequences or a sequence space. In some embodiments, the candidate sequence is a fully specified nucleotide sequence. In some embodiments, the sequence space is defined by a base template with one or more variable positions, permitted substitutions, region-level constraints, motif constraints, or codon constraints. In some embodiments, a candidate sequence comprises DNA. In some embodiments, a candidate sequence comprises RNA. In some embodiments, the candidate sequence comprises modified bases or one or more modification-state annotations.

At step 220, method 200 receives one or more design goals, target definitions, or constraints. In some embodiments, the design goal is to maximize long-term sequence stability. In some embodiments, the design goal is to maximize recognition specificity against one or more targets while reducing off-target recognition. In some embodiments, the design goal is to maximize correct copying fidelity. In some embodiments, the design goal is to jointly optimize two or more of stability, separation resistance, specificity, and fidelity. In some embodiments, the received constraints include environmental conditions such as temperature, ionic strength, solvent condition, hydration state, pH, crowding condition, or combinations thereof.

At step 230, method 200 generates or selects one or more candidate constructs for evaluation. In some embodiments, candidates are generated from an initial template by local mutation. In some embodiments, candidates are generated by codon substitution while preserving an encoded amino acid sequence. In some embodiments, candidates are generated by preserving one or more required motifs and varying flanking or noncritical positions. In some embodiments, candidates are generated in batches, streams, or iterative waves.

At step 240, method 200 determines one or more optimization metrics for the candidate constructs. In preferred embodiments, the determined metrics include a structural integrity metric, a melting or separation resistance metric, a binding specificity metric, and a replication or transcription fidelity metric. In some embodiments, one or more additional metrics are also determined. In some embodiments, the metrics are determined using one or more recognition science derived functions, one or more measured values, one or more simulated values, one or more calibrated approximations, or combinations thereof.

At step 250, method 200 ranks or selects one or more candidates based on the determined metric values. In some embodiments, the ranking is based on a composite objective. In some embodiments, the selection is based on threshold satisfaction. In some embodiments, the selected candidate is the candidate with the highest overall score. In some embodiments, the selected set includes multiple candidates representing different tradeoffs among the evaluated metrics.

At step 260, method 200 outputs one or more optimized biological sequences or constructs. In some embodiments, the output includes a single optimized sequence. In some embodiments, the output includes a ranked candidate set, an oligonucleotide library, a synthesis-ready design file, an assay plan, or a database record. In some embodiments, the output is transmitted to a synthesis platform, stored in a database, displayed to a user, or provided to another software module for downstream use.

In some embodiments, method 200 further includes an optional validation step 270 in which one or more selected candidates are synthesized, assayed, sequenced, expressed, or otherwise physically evaluated. In some embodiments, validation data from step 270 is returned to calibration or feedback engine 180 for use in updating one or more models, weights, parameters, or scoring rules.

Referring now to FIG. 3, an example descriptor framework 300 for representing a candidate biological construct is shown. In the illustrated embodiment, descriptor framework 300 includes a sequence descriptor 310, a structural descriptor 320, an energetic descriptor 330, and one or more optional additional descriptors 340. In preferred embodiments, a candidate biological construct is represented by a descriptor set D that includes at least the sequence descriptor 310, the structural descriptor 320, and the energetic descriptor 330.

In some embodiments, the candidate biological construct is represented as D=(S, H, E), where S denotes a sequence descriptor, H denotes a structural descriptor, and E denotes an energetic descriptor. In some embodiments, the representation is extended to include one or more environmental, contextual, target-specific, region-specific, or constraint-specific values. In some embodiments, the candidate biological construct is represented as D=(S, H, E, Env, T, K), where Env denotes one or more environmental descriptors, T denotes one or more target-related descriptors, and K denotes one or more constraints or control parameters.

Sequence descriptor 310 is configured to represent one or more primary sequence properties of the candidate biological construct. In some embodiments, sequence descriptor 310 includes nucleotide identities at one or more positions. In some embodiments, sequence descriptor 310 includes region annotations, motif identities, codon assignments, modification states, methylation states, barcode positions, payload positions, primer-binding positions, spacer regions, or combinations thereof. In some embodiments, sequence descriptor 310 includes one or more derived sequence values, including GC content, local motif counts, repeat counts, homopolymer lengths, or base-composition distributions.

Structural descriptor 320 is configured to represent one or more structural, conformational, or geometry-related properties associated with the candidate biological construct. In some embodiments, structural descriptor 320 includes one or more values associated with helical pitch, groove ratio, curvature, flexibility, local accessibility, secondary structure propensity, pairing probability, or combinations thereof. In some embodiments, structural descriptor 320 includes one or more local profiles defined over sequence position, spatial coordinate, or regional domain. In some embodiments, structural descriptor 320 is derived from one or more theoretical models, simulation outputs, empirical tables, or measured assay results.

Energetic descriptor 330 is configured to represent one or more energy-related, persistence-related, or interaction-related properties associated with the candidate biological construct. In some embodiments, energetic descriptor 330 includes one or more values associated with pairing energy, separation energy, coherence-related energy, thermal response, denaturation resistance, target interaction energy, off-target interaction energy, or combinations thereof. In some embodiments, energetic descriptor 330 includes one or more scalar values, one or more spectra, one or more region-specific energy values, or one or more environmental response functions.

Optional additional descriptors 340 may include one or more environmental descriptors, contextual descriptors, target descriptors, off-target descriptors, host-system descriptors, assay descriptors, polymerase descriptors, repair descriptors, chromatin descriptors, delivery descriptors, or manufacturing descriptors. In some embodiments, an environmental descriptor includes temperature, ionic strength, pH, solvent condition, hydration state, or crowding condition. In some embodiments, a target descriptor identifies one or more desired recognition targets and one or more undesired off-target species.

In some embodiments, the descriptor framework 300 permits different abstraction levels for different applications. For example, in a storage oligonucleotide embodiment, sequence descriptor 310 may emphasize payload structure, barcode structure, and primer compatibility, while structural descriptor 320 and energetic descriptor 330 emphasize persistence and separation resistance. In an aptamer embodiment, sequence descriptor 310 may emphasize motif and loop architecture, while structural descriptor 320 and energetic descriptor 330 emphasize accessibility and target-recognition behavior. In a coding-sequence embodiment, sequence descriptor 310 may emphasize synonymous codon choices and region preservation, while structural descriptor 320 and energetic descriptor 330 emphasize copy fidelity, local structure, and downstream expression compatibility.

In some embodiments, one or more values within descriptor framework 300 are directly measured. In some embodiments, one or more values are inferred from simulation. In some embodiments, one or more values are determined from recognition science derived formulas. In some embodiments, one or more values are estimated using hybrid procedures that combine measured, simulated, and theory-derived data. Accordingly, the descriptor framework 300 is not limited to any single source of parameter determination.

In some embodiments, the descriptors associated with a candidate biological construct are used as direct inputs to metric determination. In some embodiments, the descriptors are used to derive one or more intermediate values, including stability-related quantities, structural profile quantities, target interaction quantities, off-target interaction quantities, correct-event compatibility quantities, and incorrect-event compatibility quantities. In preferred embodiments, the descriptors permit the same general optimization platform to be applied across multiple biological use cases while preserving application-specific tuning and constraints.

Referring now to FIG. 4, an example metric determination framework 400 is shown. In the illustrated embodiment, metric determination framework 400 includes a structural integrity metric module 410, a melting or separation resistance metric module 420, a binding specificity metric module 430, a replication or transcription fidelity metric module 440, and an optional supplemental metric module 450. In some embodiments, outputs from one or more of metric modules 410, 420, 430, 440, and 450 are provided to scoring and ranking engine 140 for composite evaluation.

In preferred embodiments, the disclosed optimization framework evaluates each candidate biological construct according to at least four core metrics. These four core metrics include a structural integrity metric, a melting or separation resistance metric, a binding specificity metric, and a replication or transcription fidelity metric. In some embodiments, the four core metrics are sufficient to rank or select candidates. In other embodiments, one or more additional metrics are included to account for application-specific factors.

In some embodiments, one or more of the core metrics are determined using recognition science derived quantities. In some embodiments, one or more of the core metrics are determined using measured quantities, simulated quantities, empirically calibrated quantities, or combinations thereof. Accordingly, the metric framework 400 is not limited to purely theoretical determinations and may employ hybrid formulations in which recognition science derived values are combined with experimentally determined or computationally inferred values.

Structural integrity metric module 410 is configured to determine a structural integrity metric for a candidate biological construct. In general, the structural integrity metric is configured to evaluate a degree to which the candidate preserves, maintains, or approaches a desired structural or conformational state over a length, region, or coordinate domain. In some embodiments, the structural integrity metric reflects deviation from a reference profile. In some embodiments, the structural integrity metric reflects deviation from a target geometry, a target accessibility profile, a target helical state, a target folding profile, or a target persistence condition.

In some embodiments, the structural integrity metric is represented in a broad form as:

$$M\_struct = integral from 0 to L of W\_struct ( r ) * {\vert 1 - C ( r ; S , H , E , Env ) \vert}^{⋀} p dr$$

where L is a length or coordinate extent, W_struct(r) is an optional weighting function, p is greater than or equal to 1, and C(r; S, H, E, Env) is a structural profile function associated with the candidate biological construct.

In preferred embodiments, the structural integrity metric is represented by:

$$M\_struct = integral from 0 to L {of [ 1 - C ( r ; S , H ) ]}^{⋀} 2 dr$$

where C(r; S, H) represents a structural profile associated with the candidate biological construct. In some embodiments, lower values of M_struct correspond to lower deviation from a desired structure. In some embodiments, a transformed or inverted version of M_struct is used for scoring so that higher transformed values correspond to more preferred candidates.

In some embodiments, the structural profile C(r; S, H) is derived from one or more sequence-dependent or structure-dependent quantities. In some embodiments, the structural profile is derived from one or more values associated with curvature, twist, groove geometry, base-pairing propensity, loop formation tendency, accessibility, secondary structure propensity, persistence length, or combinations thereof. In some embodiments, the structural profile is determined using recognition science derived transforms. In other embodiments, the structural profile is determined using simulation outputs, measured structural data, lookup tables, machine-learning predictions, or combinations thereof.

In some embodiments, the structural integrity metric is approximated using one or more simplified forms. In a preferred simplified embodiment:

$$C ( r ; S , H ) approximately equals 1 - A * cos ( 2 * pi * r / P )$$

where A is an amplitude-related quantity and P is a periodicity-related quantity. In some embodiments:

$$A approximately equals 1 / S\_stab$$

where S_stab is a stability-related parameter. In some embodiments, the resulting structural integrity metric is approximated by:

$$M\_struct approximately equals L / ( 2 * {S\_stab}^{⋀} 2 )$$

In some embodiments, the structural integrity metric is determined using a discrete representation rather than a continuous representation. In one example:

M_struct_discrete=sum over i of w_i*[1−C_i]\hat{ }2 where C_i is a structural profile value associated with position or region i and w_i is a weighting factor. In some embodiments, the weighting factor permits increased emphasis on one or more critical positions or regions.

In some embodiments, the structural integrity metric is computed for an entire biological construct. In some embodiments, the structural integrity metric is computed for one or more subsequences, windows, domains, or annotated regions. In some embodiments, a multi-region construct includes a first region optimized for persistence, a second region optimized for recognition behavior, and a third region optimized for copying fidelity, and structural integrity metric values are computed independently or jointly across such regions.

Melting or separation resistance metric module 420 is configured to determine a metric associated with resistance to denaturation, dissociation, melting, strand separation, or related loss of stored or processable biological information. In some embodiments, the melting or separation resistance metric reflects an expected resistance of the candidate sequence to structural disruption under one or more environmental conditions. In some embodiments, the melting or separation resistance metric is used as a proxy for long-term sequence persistence.

In some embodiments, the melting or separation resistance metric is represented in a broad form as:

$$M\_melt = f\_melt ( S , H , E , Env )$$

where f_melt is a function of one or more sequence, structural, energetic, and environmental quantities. In some embodiments, the function f_melt incorporates one or more thermodynamic terms, one or more coherence-related terms, one or more interaction-energy terms, or one or more calibrated persistence terms.

In a preferred embodiment, the melting or separation resistance metric is approximated by:

$$M\_melt = S\_stab * E\_coh * lambda\_DNA$$

where S_stab is a stability-related parameter, E_coh is an energy-related parameter, and lambda_DNA is a DNA-related characteristic scale. In some embodiments, lambda_DNA is replaced by an RNA-related characteristic scale, a modified-nucleic-acid characteristic scale, or another application-appropriate characteristic scale.

In some embodiments, S_stab is determined from one or more sequence-dependent quantities. In a preferred simple embodiment:

$$S\_stab = 1.5 * ( number of CG base pairs ) + 1. * ( number of AT base pairs )$$

In some embodiments, S_stab is instead determined using sequence-context-sensitive weighting, nearest-neighbor weighting, region-specific weighting, modification-state weighting, or calibration against measured data. In some embodiments, S_stab is determined for a whole sequence. In other embodiments, S_stab is determined locally for one or more windows or subregions.

In some embodiments, E_coh is an energy-related quantity associated with coherence, structural persistence, interaction stability, or resistance to disruption. In some embodiments, E_coh is directly assigned according to a recognition science derived model. In some embodiments, E_coh is empirically calibrated. In some embodiments, E_coh is inferred from measured, simulated, or estimated energetic behavior under one or more environmental conditions.

In some embodiments, the melting or separation resistance metric is mapped to one or more practical values including a predicted melting temperature, a denaturation threshold, a dissociation threshold, a persistence score, or a degradation-resistance score. In one example:

$$T\_m \_pred = kappa\_T * M\_melt / n\_bp$$

where kappa_T is a calibration factor and n_bp is a base-pair count or equivalent sequence-length quantity. In some embodiments, such mappings are application-specific and need not be used in every embodiment.

In some embodiments, the melting or separation resistance metric depends on one or more environmental descriptors including temperature, ionic strength, pH, hydration state, solvent composition, crowding condition, strand concentration, complementary sequence availability, methylation state, or combinations thereof. Accordingly, the same candidate sequence may have different M_melt values under different intended use conditions.

Binding specificity metric module 430 is configured to determine a metric associated with selective recognition of a desired target relative to one or more undesired targets. In some embodiments, the desired target comprises a protein, a nucleic acid, a ligand, a small molecule, a cell-surface feature, a genomic locus, or a motif-bearing region. In some embodiments, an undesired target comprises one or more off-target proteins, off-target nucleic acids, off-target loci, motif analogs, homologous sequences, background species, or other non-preferred interaction partners.

In some embodiments, the binding specificity metric is represented in a broad form as:

$$M\_spec = A\_on / A\_off$$

where A_on denotes an on-target interaction quantity and A_off denotes an off-target interaction quantity. In some embodiments, A_on and A_off are amplitude-like quantities, affinity-like quantities, probability-like quantities, occupancy-like quantities, or transformed interaction measures.

In a preferred embodiment, the binding specificity metric is represented by:

$$M\_spec = \vert F\_on \vert \hat{} 2 / \vert F\_off \vert \hat{} 2$$

where F_on is an on-target interaction quantity and F_off is an off-target interaction quantity. In some embodiments, |F_on|\hat{ }2 and |F_off|\hat{ }2 represent interaction magnitudes or transformed recognition values associated with a preferred target and a non-preferred target, respectively.

In some embodiments, the binding specificity metric is extended to multiple off-targets. In one example:

$$M\_spec \_multi = \vert F\_on \vert \hat{} 2 / sum over k of \vert F\_off \_k \vert \hat{} 2$$

where F_off_k represents an interaction quantity for off-target k. In some embodiments, one or more weighting values are applied to different off-targets based on importance, abundance, risk, or other contextual relevance.

In some embodiments, on-target and off-target interaction quantities (including F_on and F_off_k) are computed from one or more implementable interaction models or measurements, including at least one of: (i) predicted binding free energy or dissociation constant for a candidate-to-target pairing; (ii) predicted occupancy, probability, or score from a sequence-motif model, position-weight matrix, or machine-learning classifier; (iii) simulated interaction outputs generated using a selected physical or statistical model; (iv) experimentally measured affinity, enrichment, cleavage rate, reporter output, or other assay-derived interaction value; or (v) a calibrated transform of any of the foregoing into a normalized interaction magnitude. In some embodiments, the same computation pathway is applied to both on-target and off-target sets so that M_spec and M_spec_multi reflect relative selectivity under the intended environmental descriptors.

In some embodiments, the binding specificity metric is computed globally over an entire candidate sequence. In some embodiments, the binding specificity metric is computed for one or more local recognition footprints. In some embodiments, different local regions are assigned different significance values. In one example, a footprint-specific metric is determined from position-wise contributions across a target binding region and compared with one or more corresponding off-target regions.

Replication or transcription fidelity metric module 440 is configured to determine a metric associated with a relative preference for correct copying, transcription, interpretation, or processing events as compared with incorrect events. In some embodiments, the fidelity metric reflects a compatibility advantage associated with a correct event relative to one or more incorrect events. In some embodiments, the fidelity metric is applicable to replication, transcription, reverse transcription, ligation, repair-template usage, guide-directed editing, or related biological processing events.

In some embodiments, the fidelity metric is represented in a broad form as:

$$M\_fidelity = Cost\_incorrect - Cost\_correct$$

where Cost_incorrect is a cost associated with one or more incorrect events and Cost_correct is a cost associated with one or more correct events. In some embodiments, higher values of M_fidelity correspond to stronger preference for correct events relative to incorrect events.

In some embodiments, one or more cost terms are determined using a recognition coverage function. In some embodiments, the recognition coverage function is represented by:

$$F\_cov ( r ) = r / ( r + X\_rec )$$

where r is a nonnegative recognition-related variable and X_rec is a recognition constant. In preferred embodiments, the overhead or cost associated with a compatibility quantity compat is expressed as:

$$Overhead\_RS ( compat ) = 1 / F\_cov ( compat )$$

which may also be written as:

$$Overhead\_RS ( compat ) = ( compat + X\_rec ) / compat$$

In a preferred embodiment, the fidelity metric is represented by:

$$M\_fidelity = X\_rec * ( 1 / compat\_incorrect - 1 / compat\_correct )$$

where compat_incorrect is an incorrect-event compatibility quantity and compat_correct is a correct-event compatibility quantity. In some embodiments, this expression is obtained from a difference between overhead values associated with correct and incorrect events.

In some embodiments, X_rec is a recognition constant used in one or more recognition science derived mappings. In a preferred embodiment, X_rec is set to phi/pi. In other embodiments, X_rec is selected, fit, calibrated, or assigned according to a measured dataset, an application type, a sequence class, an assay platform, or a model-selection rule.

In some embodiments, compat_correct and compat_incorrect are nonnegative compatibility quantities associated with correct and incorrect processing events, respectively, where larger compatibility indicates a higher predicted or measured likelihood of the corresponding event under the intended conditions. In some embodiments, compat_correct and compat_incorrect are computed from one or more of: (i) mismatch-penalty tables or stacking-compatibility tables; (ii) polymerase-, reverse-transcriptase-, ligase-, or repair-specific kinetic parameters; (iii) empirically measured misincorporation rates, error spectra, or sequencing-derived error statistics mapped into compatibility values; (iv) assay signal values or reporter outputs mapped into compatibility values; (v) simulated interaction/processing outcomes produced by a selected model; or (vi) a calibrated surrogate model trained on measured datasets. In some embodiments, compat_correct and compat_incorrect are computed using a common scale or normalization rule to support the overhead and fidelity computations described herein.

In some embodiments, the fidelity metric is aggregated across multiple possible incorrect events. In one example:

$$M\_fidelity \_set = sum over j of beta\_j * Cost\_incorrect \_j - alpha * Cost\_correct$$

where Cost_incorrect_j corresponds to incorrect event j, beta_j is an optional weighting factor, and alpha is an optional weighting factor applied to the correct event term. In some embodiments, the weighting factors permit increased emphasis on particular mismatch classes, mutation classes, transition classes, transversion classes, editing outcomes, or context-dependent error modes.

Optional supplemental metric module 450 is configured to determine one or more additional metrics that may be useful for a particular application. In some embodiments, such metrics include a manufacturability metric, a degradation-resistance metric, a secondary-structure penalty metric, an off-target aggregate penalty metric, an accessibility metric, a repair compatibility metric, an expression-related metric, an immunogenicity-related metric, a storage-density metric, a delivery-related metric, or combinations thereof.

In some embodiments, a secondary-structure penalty metric is used to penalize undesired hairpins, loops, self-dimers, cross-dimers, or other structures likely to interfere with the intended use of the candidate construct. In some embodiments, a manufacturability metric is used to penalize synthesis difficulty, low-complexity sequence patterns, repetitive motifs, homopolymer runs, cloning instability, or poor amplification behavior. In some embodiments, a degradation-resistance metric is used to account for susceptibility to hydrolysis, oxidation, enzymatic damage, fragmentation, or sequence-context-dependent deterioration.

In some embodiments, one or more additional metrics are used to capture application-specific folding or trajectory behavior. In one example, a folding-related strain quantity is represented by:

$$local\_strain = \vert d - 1 \vert$$

where d is a distance or separation quantity between neighboring encoded states. In some embodiments, an aggregated folding-related strain quantity is represented by:

$$trajectory\_strain = sum over adjacent states of local\_strain$$

In some embodiments, one or more such quantities are incorporated into a supplemental metric for coding-sequence optimization, structure-aware sequence optimization, or downstream realization-aware optimization.

In some embodiments, one or more of the foregoing metrics are determined independently. In some embodiments, one or more metrics share one or more intermediate values. For example, one or more stability-related quantities may contribute to both the structural integrity metric and the melting or separation resistance metric. Likewise, one or more compatibility-related quantities may contribute to both the binding specificity metric and the replication or transcription fidelity metric.

Accordingly, the disclosed framework permits both modular and integrated metric determination. In some embodiments, the metric values are left in their native forms for downstream ranking. In some embodiments, one or more metric values are normalized, transformed, bounded, inverted, or otherwise mapped prior to composite scoring. In some embodiments, transformations are selected so that larger transformed values correspond to more desirable candidate behavior. In some embodiments, monotone-equivalent transformations are treated as functionally equivalent for ranking purposes.

In some embodiments, the metric framework 400 is configured so that different applications emphasize different subsets of the available metrics. For example, a storage-optimized embodiment may emphasize structural integrity and separation resistance. An aptamer or biosensor embodiment may emphasize specificity and accessibility. A coding-sequence embodiment may emphasize copying fidelity and local structure. A guide-sequence embodiment may emphasize target specificity, off-target suppression, and compatibility with editing or processing pathways.

In some embodiments, one or more metric values are determined for a whole construct, one or more metric values are determined for one or more local regions, and the resulting values are combined according to one or more region-specific rules. In this manner, a multi-region biological construct may be optimized so that different segments satisfy different functional goals while still being evaluated within a common recognition-based framework.

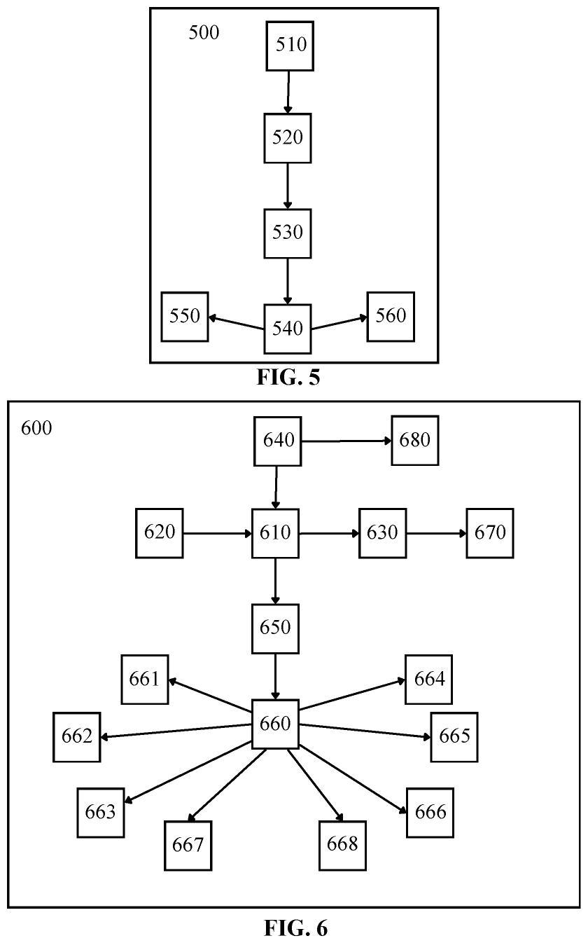

Referring now to FIG. 5, an example composite scoring and candidate selection process 500 is shown. In the illustrated embodiment, process 500 includes a metric intake stage 510, a normalization or transformation stage 520, a weighting stage 530, a composite scoring stage 540, a filtering or threshold stage 550, and a candidate ranking and selection stage 560.

In some embodiments, composite scoring and candidate selection process 500 receives as inputs one or more metric values determined for each candidate biological construct. In preferred embodiments, the received metric values include at least a structural integrity metric, a melting or separation resistance metric, a binding specificity metric, and a replication or transcription fidelity metric. In some embodiments, one or more supplemental metric values are also received.

Metric intake stage 510 is configured to receive raw metric values, partially processed metric values, or region-specific metric values associated with one or more candidates. In some embodiments, the received values are stored together with candidate identifiers, sequence annotations, region annotations, target identifiers, off-target identifiers, environmental settings, or constraint flags. In some embodiments, metric intake stage 510 rejects candidates that fail one or more hard constraints before further scoring is performed.

Normalization or transformation stage 520 is configured to convert one or more metric values into forms suitable for comparison or aggregation. In some embodiments, normalization or transformation stage 520 applies identity mappings, reciprocal mappings, logarithmic mappings, bounded mappings, rank-based mappings, min-max normalization, percentile normalization, z-score normalization, or combinations thereof. In some embodiments, the selected transformation is chosen so that a higher transformed value corresponds to a more desirable candidate.

In some embodiments, the structural integrity metric is transformed by an inverse or reciprocal mapping so that lower structural deviation produces a higher transformed score. In one example:

$$N\_struct = 1 / ( M\_struct + epsilon )$$

where epsilon is a nonzero stabilizing quantity. In some embodiments, epsilon is selected to avoid singularity or excessive sensitivity for small metric values.

In some embodiments, the melting or separation resistance metric is used directly or with a monotone transformation. In one example:

$$N\_melt = log ( 1 + M\_melt )$$

In other embodiments, the melting or separation resistance metric is normalized relative to a reference distribution, a target threshold, or an application-specific scale.

In some embodiments, the binding specificity metric is used directly, log-transformed, clipped, bounded, or normalized relative to one or more target and off-target panels. In one example:

$$N\_spec = log ( 1 + M\_spec )$$

In another example, a bounded transformation is used so that extremely large specificity values do not dominate the composite score.

In some embodiments, the replication or transcription fidelity metric is used directly or transformed into a bounded or normalized fidelity score. In one example:

N_fidelity=M_fidelity/(M_fidelity+k_f)where k_f is a scale-setting quantity. In some embodiments, k_f is selected based on a target application, an assay range, or an empirical calibration set.

Weighting stage 530 is configured to assign relative importance to two or more normalized or transformed metric values. In some embodiments, weighting stage 530 applies fixed weights. In some embodiments, weighting stage 530 applies user-selected weights. In some embodiments, weighting stage 530 applies application-specific default weights. In some embodiments, weighting stage 530 applies weights learned or updated from measured data, simulation data, historical performance data, or combinations thereof.

In some embodiments, weighting stage 530 assigns a first weight to structural integrity, a second weight to melting or separation resistance, a third weight to binding specificity, and a fourth weight to replication or transcription fidelity. In some embodiments, additional weights are assigned to one or more supplemental metrics. In some embodiments, one or more weights are region-specific, target-specific, assay-specific, or environment-specific.

Composite scoring stage 540 is configured to determine a composite score for each candidate. In some embodiments, the composite score is represented in a broad form as:

$$Score = sum over i of w\_i * N\_i ( M\_i )$$

where M_i denotes a metric value, N_i denotes an optional normalization or transformation, and w_i denotes a corresponding weight.

In a preferred embodiment, the composite score is represented by: Score=w1(1/(M_struct+epsilon))+w2M_melt+w3M_spec+w4M_fidelity**, where epsilon is a nonzero stabilizing quantity. In some embodiments, one or more terms are replaced by transformed, bounded, normalized, or calibrated counterparts as described herein.

In some embodiments, the composite score is further modified by one or more penalty terms. In one example:

$$Score\_adj = Score - P\_soft$$

where P_soft represents one or more soft-constraint penalties. In some embodiments, P_soft includes penalties for synthesis difficulty, forbidden motifs, excessive homopolymers, undesired secondary structure, unacceptable off-target exposure, unacceptable immunogenicity, or combinations thereof.

In some embodiments, the composite score is further modified by one or more bonus terms. In one example:

$$Score\_final = Score\_adj + B\_pref$$

where B_pref represents one or more preference bonuses associated with desirable optional features, including preferred codons, preferred motifs, preferred accessibility states, preferred storage density, preferred manufacturability, or combinations thereof.

In some embodiments, filtering or threshold stage 550 is configured to apply one or more decision rules before final ranking or selection. In some embodiments, filtering or threshold stage 550 removes any candidate having a metric value below or above a threshold, depending on the direction of desirability for the metric. In some embodiments, filtering or threshold stage 550 removes any candidate having an off-target quantity above a permitted threshold, a fidelity quantity below a required threshold, a secondary-structure penalty above a permitted threshold, or a manufacturability value below a required threshold.

In one non-limiting example, filtering or threshold stage 550 applies a multi-metric acceptance rule in which a candidate is retained only if: (i) M_spec_multi≥T_spec, (ii) M_fidelity≥T_fid, and (iii) M_struct≤T_struct, where T_spec, T_fid, and T_struct are user-specified or application-default thresholds. Candidates failing any required threshold are rejected, and the remaining candidates are ranked by the composite score (or Pareto selection) as described herein.

By way of example, T_spec may be set in a range from 2 to 100, T_fid may be set in a range from 0.01 to 10, and T_struct may be set in a range selected based on sequence length L.

In some embodiments, filtering or threshold stage 550 applies a hard-threshold rule to one or more primary metrics and a soft-threshold rule to one or more supplemental metrics. In some embodiments, a candidate must satisfy all hard thresholds to remain under consideration. In some embodiments, failure to satisfy a soft threshold results in a penalty rather than immediate rejection.

Candidate ranking and selection stage 560 is configured to rank, sort, cluster, filter, or otherwise select one or more candidates for output. In some embodiments, stage 560 selects a single highest-ranked candidate. In some embodiments, stage 560 selects a top-N candidate list. In some embodiments, stage 560 selects all candidates satisfying one or more threshold conditions. In some embodiments, stage 560 selects one or more candidates from a Pareto-optimal set.

In some embodiments, candidate ranking and selection stage 560 uses Pareto selection rather than or in addition to scalar composite scoring. In such embodiments, a candidate may be retained if it is not dominated by another candidate across a selected set of metrics. In some embodiments, Pareto selection is useful where the candidate set contains meaningful tradeoffs among persistence, specificity, fidelity, and other application-specific objectives.

In some embodiments, composite scoring and candidate selection process 500 is performed on a candidate-by-candidate basis. In some embodiments, process 500 is performed in batches. In some embodiments, process 500 is performed iteratively as new candidates are generated or as new validation data becomes available. In some embodiments, scoring rules, normalization rules, or weights are updated between iterations.

In some embodiments, candidate selection is performed at the level of an entire construct. In some embodiments, candidate selection is performed at the level of one or more local regions, followed by assembly of a full construct from selected local solutions. In some embodiments, a first region is selected according to one metric emphasis, a second region is selected according to a different metric emphasis, and a full construct is then evaluated according to a global score.

In some embodiments, a multi-region score is represented by:

$$Score\_multi = sum over regions j of q\_j * Score\_j$$

where Score_j is a score associated with region j and q_j is a region-level weighting factor. In some embodiments, one or more region-level weighting factors are determined according to functional importance, sequence length, biological role, or user selection.

In some embodiments, composite scoring and candidate selection process 500 includes diversity-aware selection. In some embodiments, where multiple candidates have similar scores, a subset is selected so as to preserve sequence diversity, motif diversity, target-space diversity, or region-pattern diversity. In some embodiments, such diversity-aware selection is useful for library generation, assay panels, screening sets, or exploratory validation workflows.

In some embodiments, process 500 includes uncertainty-aware selection. In some embodiments, one or more metric values are associated with confidence ranges, posterior estimates, error bars, or uncertainty scores. In some embodiments, candidate ranking accounts for both predicted performance and associated uncertainty. In some embodiments, candidates having slightly lower predicted scores but significantly higher confidence may be preferred for validation or production.

In some embodiments, the composite scoring and candidate selection process 500 is configured differently for different application classes. In a storage-oriented embodiment, the process may emphasize structural integrity and melting or separation resistance. In an aptamer or biosensor embodiment, the process may emphasize specificity and accessibility. In a coding-sequence embodiment, the process may emphasize fidelity, manufacturability, and local structural acceptability. In a guide-sequence embodiment, the process may emphasize specificity, off-target suppression, and processing compatibility.

In some embodiments, process 500 produces one or more output records that include not only selected candidates but also supporting decision information. In some embodiments, the output record includes raw metric values, transformed metric values, applied weights, penalty terms, threshold flags, region-level scores, target and off-target context, and a rationale code indicating why a given candidate was selected or rejected.

In some embodiments, process 500 is configured so that candidate selection remains robust under equivalent mathematical reformulations. For example, reciprocal, bounded, normalized, shifted, or log-transformed variants of the same metric may be substituted without changing the underlying functional ranking objective, provided that the transformed values preserve the intended order or tradeoff structure. Accordingly, the disclosed candidate selection framework is not limited to any single exact scalarization or transformation form.

In some embodiments, process 500 provides selected candidates directly to output interface 150 for synthesis, storage, display, export, assay planning, or downstream computational use. In some embodiments, process 500 provides one or more partially selected candidates to a further refinement stage in which additional local search, assay-guided optimization, or application-specific screening is performed before final output.

Referring now to FIG. 6, an example computing environment 600 for implementing the disclosed methods is shown. In the illustrated embodiment, computing environment 600 includes one or more processors 610, a memory 620, a storage subsystem 630, a communication interface 640, a user interface 650, and one or more software modules 660. In some embodiments, computing environment 600 further includes an accelerator subsystem 670 and one or more external resource connections 680.

Processor 610 is configured to execute instructions associated with candidate generation, metric determination, scoring, ranking, selection, output generation, calibration, feedback processing, or combinations thereof. In some embodiments, processor 610 includes one or more central processing units. In some embodiments, processor 610 includes one or more graphics processing units, tensor processing units, field-programmable gate arrays, application-specific integrated circuits, or other specialized computation devices. In some embodiments, two or more processors cooperate in a distributed or parallel computation framework.

Memory 620 is configured to store executable instructions, working data, candidate sequences, intermediate metric values, parameter values, target definitions, off-target definitions, transformation rules, weighting values, or combinations thereof. In some embodiments, memory 620 includes volatile memory. In some embodiments, memory 620 includes non-volatile memory. In some embodiments, memory 620 stores temporary candidate sets during iterative optimization and stores region-level results for subsequent construct assembly or final ranking.

Storage subsystem 630 is configured to store sequence databases, candidate libraries, prior optimization histories, assay results, calibration data, model parameters, export files, and other persistent information used by the disclosed platform. In some embodiments, storage subsystem 630 includes local storage. In some embodiments, storage subsystem 630 includes remote or network-accessible storage. In some embodiments, storage subsystem 630 stores multiple versions of a candidate construct and associated score records so that candidate lineage, optimization changes, and validation results may be tracked over time.

Communication interface 640 is configured to permit data exchange between computing environment 600 and one or more external systems. In some embodiments, communication interface 640 supports communication with sequence databases, synthesis vendors, assay platforms, cloud computation systems, laboratory information systems, genomic databases, model repositories, or combinations thereof. In some embodiments, communication interface 640 supports secure transmission of sequence files, ranked outputs, calibration data, or assay results.

User interface 650 is configured to permit a user to provide one or more candidate sequences, constraints, target definitions, metric preferences, region annotations, application settings, threshold values, or weighting values, and to receive one or more outputs generated by the system. In some embodiments, user interface 650 includes a graphical user interface. In some embodiments, user interface 650 includes a command-line interface, an application programming interface, a web interface, or a combination thereof.

Software modules 660 are configured to implement one or more aspects of the disclosed optimization workflow. In some embodiments, software modules 660 include an input module 661, a candidate generation module 662, a descriptor module 663, a metric module 664, a scoring module 665, a selection module 666, an output module 667, and a calibration module 668. In some embodiments, one or more of software modules 660 correspond to the functional engines described with reference to system 100. In some embodiments, one or more of software modules 660 are combined. In some embodiments, one or more of software modules 660 are separated into additional submodules.

Input module 661 is configured to receive and parse candidate sequence information, region maps, environmental settings, assay-derived values, target definitions, off-target definitions, or other input data. In some embodiments, input module 661 validates syntax, format, sequence alphabet, region annotation consistency, and application-specific requirements before the candidate sequence information is passed to downstream modules.

Candidate generation module 662 is configured to generate, mutate, edit, or otherwise define candidate biological constructs for evaluation. In some embodiments, candidate generation module 662 performs template expansion, codon substitution, motif-preserving mutation, constrained sequence enumeration, stochastic exploration, evolutionary search, or combinations thereof. In some embodiments, candidate generation module 662 operates jointly with one or more hard or soft constraint routines.

Descriptor module 663 is configured to derive, assign, or retrieve one or more descriptor values associated with each candidate biological construct. In some embodiments, descriptor module 663 determines sequence descriptors, structural descriptors, energetic descriptors, environmental descriptors, target descriptors, off-target descriptors, or combinations thereof. In some embodiments, descriptor module 663 retrieves precomputed values from storage subsystem 630 or external resource connections 680. In some embodiments, descriptor module 663 computes one or more intermediate values needed by metric module 664.

Metric module 664 is configured to determine one or more metric values for each candidate biological construct. In preferred embodiments, metric module 664 determines a structural integrity metric, a melting or separation resistance metric, a binding specificity metric, and a replication or transcription fidelity metric. In some embodiments, metric module 664 also determines one or more supplemental metrics. In some embodiments, metric module 664 uses theory-derived, measured, simulated, calibrated, or hybrid procedures.

Scoring module 665 is configured to normalize, transform, weight, combine, or otherwise process metric values into one or more scores, threshold flags, or ranking signals. In some embodiments, scoring module 665 applies reciprocal transforms, bounded transforms, logarithmic transforms, region-level weighting, thresholding rules, penalty terms, bonus terms, Pareto logic, or combinations thereof. In some embodiments, scoring module 665 produces both whole-construct and region-level scores.

Selection module 666 is configured to rank, filter, cluster, diversify, or otherwise select one or more candidate constructs based on outputs from scoring module 665. In some embodiments, selection module 666 selects a single highest-ranked candidate. In some embodiments, selection module 666 selects a library or candidate panel. In some embodiments, selection module 666 selects a Pareto front or a diversity-preserving subset for validation.

Output module 667 is configured to generate one or more output formats for downstream use. In some embodiments, output module 667 produces a sequence file, a ranked report, a synthesis-ready construct definition, a design summary, an assay plan, a library file, a machine-readable export, a database record, or combinations thereof. In some embodiments, output module 667 transmits one or more selected candidates directly to an external synthesis or assay platform.

Calibration module 668 is configured to receive measured, simulated, inferred, or user-provided data and to update one or more parameters used in the disclosed workflow. In some embodiments, calibration module 668 updates normalization ranges, threshold values, metric mappings, recognition constants, weighting values, region preferences, model-selection rules, or correction terms. In some embodiments, calibration module 668 is used in a closed-loop workflow in which experimental results from previously selected candidates influence the evaluation of later candidates.

Accelerator subsystem 670, when present, is configured to accelerate one or more computational tasks associated with candidate generation, descriptor determination, metric computation, large-library screening, matrix operations, machine-learning inference, or combinations thereof. In some embodiments, accelerator subsystem 670 includes one or more graphics processing units or tensor processing units. In some embodiments, accelerator subsystem 670 supports large-batch ranking of candidate sequences or rapid evaluation of variant libraries.

External resource connections 680, when present, are configured to permit access to one or more external systems including genomic references, codon usage tables, motif repositories, target libraries, off-target libraries, thermodynamic tables, machine-learning models, assay databases, synthesis vendor systems, cloud compute clusters, or combinations thereof. In some embodiments, external resource connections 680 allow the disclosed platform to dynamically retrieve context-specific information relevant to a particular biological application.

In some embodiments, computing environment 600 is implemented on a single workstation, server, or cloud instance. In some embodiments, computing environment 600 is distributed across multiple computing nodes. In some embodiments, one or more software modules are executed locally while one or more resource-intensive tasks are executed remotely. In some embodiments, different portions of the workflow are assigned to different processors or different machines according to throughput, latency, security, or data-locality requirements.

In some embodiments, computing environment 600 supports batch evaluation of large candidate libraries. In some embodiments, computing environment 600 supports interactive optimization of a single candidate sequence or a small set of candidate sequences. In some embodiments, computing environment 600 supports streaming or iterative workflows in which additional candidate sequences are generated or received over time and are incorporated into an ongoing ranking or selection process.

In some embodiments, computing environment 600 supports role-based or user-based control over the optimization process. For example, a first user may define one or more biological objectives, a second user may define synthesis or manufacturing constraints, and a third user may review ranked output candidates for validation planning. In some embodiments, access privileges are used to limit who may edit sequence templates, target definitions, off-target panels, or scoring rules.

In some embodiments, computing environment 600 stores or generates audit information associated with the optimization process. In some embodiments, such audit information includes timestamps, input versions, candidate lineage data, metric versions, parameter versions, model selections, threshold settings, output versions, or combinations thereof. In some embodiments, such audit information facilitates traceability from a selected biological construct back to the conditions and computations under which the construct was selected.

In some embodiments, computing environment 600 supports integration with laboratory workflows. In some embodiments, output module 667 or communication interface 640 transmits selected sequence candidates, library definitions, primer definitions, cloning instructions, or assay plans to one or more external systems used for synthesis, validation, sequencing, expression analysis, or storage preparation. In some embodiments, assay results are returned electronically and stored in storage subsystem 630 for later use by calibration module 668.

In some embodiments, computing environment 600 supports application-specific configuration sets. A storage-oriented configuration may emphasize persistence, separation resistance, and library export. A specificity-oriented configuration may emphasize target panels, off-target panels, and accessibility analysis. A fidelity-oriented configuration may emphasize compatibility values, error models, and copying or editing workflows. Accordingly, computing environment 600 is not limited to any single application mode.

In some embodiments, one or more of the disclosed software modules are embodied in a non-transitory computer-readable medium storing instructions that, when executed by one or more processors, cause the one or more processors to perform any of the methods described herein. In some embodiments, one or more software modules are embodied in cloud-deployed services, local executables, containerized services, embedded logic, distributed microservices, or combinations thereof.

In some embodiments, the computing environment 600 is used to generate optimized biological sequences that are then physically synthesized, cloned, amplified, transcribed, packaged, stored, screened, assayed, or otherwise implemented. Accordingly, the disclosed computing environment is not limited to abstract data processing and is expressly directed to practical generation and selection of biological constructs for real-world use.

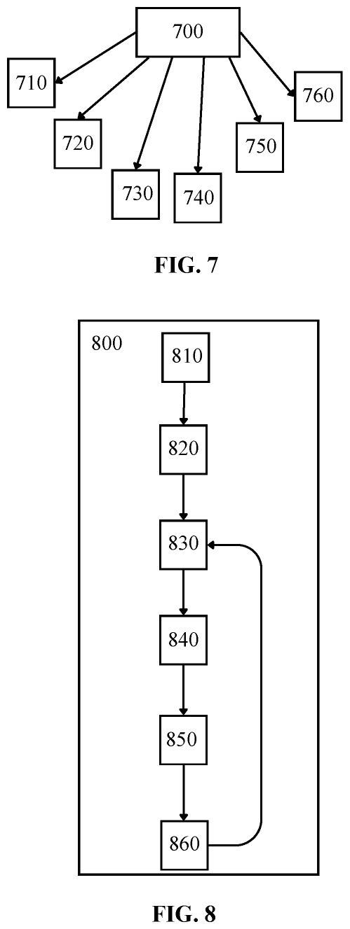

Referring now to FIG. 7, an example application embodiment framework 700 is shown. In the illustrated embodiment, framework 700 depicts use of the disclosed optimization platform across multiple biological sequence-design applications. In some embodiments, framework 700 includes a nucleic acid data storage embodiment 710, an aptamer or probe design embodiment 720, a biosensor design embodiment 730, a coding-sequence optimization embodiment 740, a regulatory sequence optimization embodiment 750, and a genome-editing-related embodiment 760. In some embodiments, two or more of such embodiments are implemented using a common underlying metric and ranking architecture.

In some embodiments, the disclosed optimization platform is configured so that different biological applications use different subsets of candidate descriptors, different subsets of optimization metrics, different weighting schemes, different constraints, or combinations thereof, while still operating within the same general recognition science derived framework. In this manner, the invention provides a unified architecture capable of supporting multiple types of biological information storage and processing tasks without requiring separate unrelated design engines for each task.

The embodiments described with respect to storage, aptamers, biosensors, coding optimization, regulatory optimization, and editing workflows share a common inventive concept: a computer-implemented architecture that represents candidates using a descriptor set and evaluates candidates using at least the four core metrics, including fidelity determined via recognition-coverage and overhead functions, and selects candidates using composite scoring, thresholding, or Pareto logic within a common optimization engine.

In nucleic acid data storage embodiment 710, a candidate biological construct comprises one or more storage oligonucleotides, storage regions, indexing regions, barcode regions, primer regions, parity regions, or payload regions. In some embodiments, the design goal is to maximize long-term structural integrity, separation resistance, retrieval robustness, or combinations thereof. In some embodiments, the design goal further includes minimizing degradation susceptibility, minimizing synthesis difficulty, or maintaining compatibility with amplification and readout workflows.

In some embodiments of nucleic acid data storage embodiment 710, the structural integrity metric and the melting or separation resistance metric are assigned greater weights than the binding specificity metric. In some embodiments, the binding specificity metric is still used to reduce unintended cross-hybridization among storage constructs, primer regions, barcode regions, or retrieval probes. In some embodiments, the fidelity metric is used to favor storage designs associated with improved copying, amplification, sequencing, or error-correction performance.

In some embodiments of nucleic acid data storage embodiment 710, the disclosed platform evaluates a library of candidate storage sequences and outputs one or more ranked storage constructs or storage pools. In some embodiments, the output includes sequence records, storage scores, retrieval scores, primer compatibility data, expected persistence values, or combinations thereof. In some embodiments, the output is transmitted to a synthesis workflow for manufacture of a storage library.

In aptamer or probe design embodiment 720, a candidate biological construct comprises a nucleic acid sequence intended to bind a desired target species. In some embodiments, the desired target species comprises a protein, peptide, nucleic acid, small molecule, metabolite, ion, marker, or other molecule of interest. In some embodiments, one or more off-target species are defined and used in the specificity analysis.

In some embodiments of aptamer or probe design embodiment 720, the binding specificity metric is assigned a greater weight than one or more other core metrics. In some embodiments, the structural integrity metric is also used to favor candidates that preserve a loop architecture, stem-loop architecture, accessible motif architecture, or other target-compatible structure. In some embodiments, the melting or separation resistance metric is used where assay stability, environmental robustness, or shelf stability is relevant.

In some embodiments, aptamer or probe design embodiment 720 is used to select a single optimized aptamer candidate. In some embodiments, aptamer or probe design embodiment 720 is used to select a ranked panel of candidates for laboratory screening. In some embodiments, selected candidates are synthesized and evaluated by one or more binding assays, and the measured results are returned to the calibration workflow for later refinement.

In biosensor design embodiment 730, a candidate biological construct comprises one or more sensing elements, recognition sequences, reporter-coupled sequences, switchable nucleic acid structures, or target-responsive motifs. In some embodiments, a biosensor candidate is optimized for target recognition, dynamic response, signal discrimination, structural stability, environmental tolerance, or combinations thereof. In some embodiments, the candidate construct further includes one or more linker regions, substrate-coupled regions, immobilization regions, or signal-output regions.

In some embodiments of biosensor design embodiment 730, specificity is evaluated against one or more chemically or structurally related off-target species. In some embodiments, structural integrity is evaluated with respect to a desired sensing conformation or switching conformation. In some embodiments, the disclosed platform outputs one or more biosensor sequence candidates together with expected response rankings, predicted selectivity values, or recommended validation assays.

In coding-sequence optimization embodiment 740, a candidate biological construct comprises a coding region that encodes a desired amino acid sequence. In some embodiments, the candidate coding region is generated by synonymous codon substitution while preserving the encoded protein sequence. In some embodiments, the design goal includes increasing copying fidelity, reducing undesirable local structure, improving synthesis or amplification behavior, improving persistence, or combinations thereof.

In some embodiments of coding-sequence optimization embodiment 740, one or more codon-preservation or amino-acid-preservation constraints are treated as hard constraints. In some embodiments, the fidelity metric is assigned a substantial weight to favor candidate coding sequences associated with improved correct-event preference during replication, transcription, reverse transcription, or related processing. In some embodiments, one or more supplemental metrics are used to reduce repetitive sequence patterns, excessive GC extremes, strong undesired secondary structures, or synthesis-limiting motifs.

In some embodiments, coding-sequence optimization embodiment 740 further includes one or more downstream realization-aware or folding-aware metrics. In some embodiments, such metrics are used to favor codon trajectories or local sequence choices expected to reduce downstream strain, improve realizability, or improve compatibility with later expression or folding processes. In some embodiments, one or more such quantities are used as supplemental metrics in the composite score.

In regulatory sequence optimization embodiment 750, a candidate biological construct comprises a promoter sequence, enhancer sequence, operator sequence, transcription-factor binding site, repressor site, activator site, untranslated region, splicing-related sequence, insulator sequence, or other regulatory element. In some embodiments, the design goal includes improving target recognition specificity, preserving or enhancing accessibility, reducing off-target recognition, maintaining structural suitability, or combinations thereof.

In some embodiments of regulatory sequence optimization embodiment 750, the specificity metric is used to compare a desired regulatory target interaction against one or more non-preferred interactions. In some embodiments, the structural integrity metric is used to preserve a preferred local architecture or accessibility profile. In some embodiments, a region-specific analysis is used so that core motif positions, flanking positions, and spacer positions are optimized under different rules or weights.

In genome-editing-related embodiment 760, a candidate biological construct comprises a guide sequence, a donor template, a repair template, a guide-associated scaffold region, a prime-editing-related sequence, an RNA-guided recognition region, or combinations thereof. In some embodiments, the design goal includes improving on-target specificity, reducing off-target interactions, improving correct editing compatibility, improving template persistence, or combinations thereof.

In some embodiments of genome-editing-related embodiment 760, the specificity metric is used to compare a desired genomic target interaction with a set of possible off-target interactions. In some embodiments, the fidelity metric is used to favor candidates associated with improved correct-event preference in the editing, repair, copying, or guide-directed processing context. In some embodiments, the melting or separation resistance metric is used to improve stability of donor templates, guide constructs, or related components under intended use conditions.

In some embodiments, the disclosed platform additionally supports RNA design applications. In some embodiments, an RNA design application includes mRNA optimization, noncoding RNA optimization, antisense design, small interfering RNA design, ribozyme design, or RNA aptamer design. In some embodiments, an RNA design application uses one or more RNA-specific structural descriptors, energetic descriptors, environmental descriptors, or process-compatibility descriptors while still employing one or more of the core metrics disclosed herein.

In some embodiments, the disclosed platform additionally supports modified-nucleic-acid design applications. In some embodiments, a modified-nucleic-acid design application includes methylated DNA, chemically modified RNA, backbone-modified oligonucleotides, nucleic acid analogs, or combinations thereof. In some embodiments, one or more descriptors or metric mappings are adjusted to account for modified-base behavior, altered stability, altered processing compatibility, altered recognition behavior, or altered delivery or persistence characteristics.

In some embodiments, the disclosed platform supports multi-region constructs that combine two or more of the foregoing application types within a single biological construct. For example, a construct may include a storage region together with a primer region, a coding region together with a regulatory region, a guide region together with a scaffold region, or an aptamer region together with a reporter-coupled region. In some embodiments, different regions are scored using different weights or different local metrics before a full-construct score is determined.

In some embodiments, application embodiment framework 700 is used to configure default weighting values, target definitions, environmental assumptions, output formats, or validation workflows for a selected application class. In some embodiments, a storage configuration emphasizes persistence and separation resistance, an aptamer configuration emphasizes specificity and structural suitability, a coding configuration emphasizes fidelity and manufacturability, and an editing configuration emphasizes specificity and correct-event compatibility.

In some embodiments, one or more application embodiments are implemented sequentially. For example, a candidate coding sequence may first be optimized under coding-sequence optimization embodiment 740 and then evaluated under regulatory sequence optimization embodiment 750 if the coding sequence is to be incorporated into a larger expression construct. In some embodiments, a donor template may be optimized under genome-editing-related embodiment 760 and then further evaluated under data-storage-like persistence criteria if long-term storage of the donor template is also desired.

In some embodiments, one or more application embodiments are implemented concurrently. In such embodiments, a candidate construct may be scored simultaneously under two or more application-specific objective sets, and a final selection may be based on a combined or constrained optimization rule. In some embodiments, such concurrent evaluation is useful for multifunctional constructs, hybrid assay constructs, or constructs intended for multiple downstream workflows.

In some embodiments, framework 700 demonstrates that the disclosed optimization platform is not limited to any single biological niche. Rather, the same general recognition science derived optimization architecture may be adapted to multiple sequence-design contexts by varying descriptors, targets, constraints, metric weights, or output requirements, while preserving a common underlying candidate evaluation and selection process.

In some embodiments, one or more outputs of framework 700 include one or more optimized biological sequences, one or more ranked candidate panels, one or more synthesis-ready designs, one or more validation plans, one or more assay recommendations, one or more database entries, or combinations thereof. In some embodiments, selected outputs are physically synthesized, cloned, amplified, transcribed, packaged, screened, stored, or otherwise implemented in a laboratory, manufacturing, diagnostic, therapeutic, or storage context.

The foregoing application embodiments are illustrative and non-limiting. Additional embodiments may include sequence library generation, genome stabilization workflows, persistence-oriented therapeutic nucleic acid design, environmental robustness optimization, repair-oriented template design, or other biological information storage and processing applications that make use of one or more of the disclosed descriptors, metrics, scoring routines, and candidate selection processes.

Referring now to FIG. 8, an example validation and output workflow 800 is shown. In the illustrated embodiment, workflow 800 includes an optimized candidate output stage 810, a library or construct generation stage 820, an assay planning stage 830, a physical implementation stage 840, a measurement and evaluation stage 850, and a feedback and record stage 860.

In some embodiments, optimized candidate output stage 810 receives one or more selected candidates from the scoring and ranking workflow and prepares the selected candidates for downstream use. In some embodiments, the output includes a single selected biological sequence. In some embodiments, the output includes a ranked candidate list, a diversity-preserved candidate subset, a top-N panel, a Pareto-selected set, or a region-assembled construct definition. In some embodiments, the output further includes metric values, transformed values, weights, threshold flags, region annotations, target and off-target information, or combinations thereof.

In some embodiments, library or construct generation stage 820 is configured to generate one or more practical output forms from the selected candidates. In some embodiments, such output forms include oligonucleotide libraries, pooled candidate libraries, vector-ready constructs, synthesis-ready files, cloning plans, primer definitions, donor-template files, guide libraries, aptamer panels, storage-sequence libraries, or combinations thereof. In some embodiments, library or construct generation stage 820 converts internal selection records into formats accepted by synthesis systems, laboratory systems, or downstream computational tools.

In some embodiments, assay planning stage 830 is configured to determine one or more validation workflows for one or more selected candidates. In some embodiments, the selected assay depends on the application context. For example, a storage-oriented candidate may be assigned persistence testing, amplification testing, or retrieval testing. An aptamer candidate may be assigned target-binding and off-target-binding assays. A coding-sequence candidate may be assigned synthesis evaluation, copy-fidelity evaluation, expression evaluation, or structure-related evaluation. An editing-related candidate may be assigned on-target and off-target assays, editing outcome assays, or repair-template usage assays.

In some embodiments, assay planning stage 830 generates one or more assay recommendations based on the metrics that most strongly influenced selection. In some embodiments, a candidate with a high specificity score is assigned one or more binding or discrimination assays. In some embodiments, a candidate with a high melting or persistence score is assigned thermal challenge or storage challenge assays. In some embodiments, a candidate selected substantially on the basis of copying fidelity is assigned one or more misincorporation, sequencing, replication, or transcription assays.

In some embodiments, physical implementation stage 840 includes chemical synthesis, enzymatic synthesis, cloning, amplification, transcription, formulation, packaging, immobilization, storage preparation, library pooling, or combinations thereof. In some embodiments, the selected biological construct is physically produced as a nucleic acid molecule or library. In some embodiments, the selected biological construct is incorporated into a vector, assay substrate, storage medium, diagnostic device, editing workflow, cellular system, or other physical context.

In some embodiments, measurement and evaluation stage 850 includes one or more laboratory assays, instrument readouts, sequencing workflows, expression measurements, persistence measurements, binding measurements, editing measurements, or combinations thereof. In some embodiments, measurement and evaluation stage 850 produces one or more observed values corresponding to structural integrity, denaturation resistance, recognition specificity, fidelity, manufacturability, degradation resistance, or other selected performance variables.

In some embodiments, measurement and evaluation stage 850 compares observed values with predicted values generated during the earlier optimization workflow. In some embodiments, this comparison is used to assess model adequacy, candidate robustness, threshold calibration, or the need for application-specific adjustments. In some embodiments, the comparison is performed at the level of an entire construct. In some embodiments, the comparison is performed at the level of one or more regions, motifs, or interaction sites.

In some embodiments, feedback and record stage 860 is configured to store, transmit, or otherwise use measured results. In some embodiments, stage 860 records measured assay values together with the associated candidate identities, predicted metric values, parameter settings, environmental conditions, timestamps, or combinations thereof. In some embodiments, such records are stored in one or more databases for later retrieval, benchmarking, or calibration.

In some embodiments, feedback and record stage 860 transmits measured results to a calibration workflow so that one or more metric mappings, weighting values, normalization rules, threshold values, correction terms, or target-specific models may be updated. In some embodiments, this permits iterative refinement of candidate generation, scoring, or ranking based on experimentally observed outcomes. In some embodiments, repeated iterations improve performance for a selected application class, target family, sequence family, assay system, or environmental condition.

In some embodiments, the disclosed validation and output workflow 800 supports closed-loop optimization. In some embodiments, an initial set of selected candidates is produced computationally, a subset is physically implemented and measured, and the resulting measurement data is used to guide later rounds of candidate generation and selection. In some embodiments, this closed-loop process permits progressive refinement of sequence libraries, target-specific designs, region-specific solutions, or construct architectures.

In some embodiments, validation and output workflow 800 supports both single-candidate deployment and library-based deployment. In some embodiments, a single candidate is selected for direct production and use. In some embodiments, a ranked set or diversity-preserved subset is selected for screening, multiplex use, or library storage. In some embodiments, such library-based deployment is particularly useful in aptamer discovery, probe design, storage-sequence development, guide-sequence development, or candidate panel benchmarking.

In some embodiments, the output of workflow 800 includes one or more machine-readable files. In some embodiments, such files include sequence tables, sequence library files, assay scheduling files, synthesis order files, construct assembly files, database upload files, or combinations thereof. In some embodiments, such machine-readable files enable automated transfer of selected outputs to other systems without manual reformulation.

In some embodiments, the output of workflow 800 includes one or more human-readable reports. In some embodiments, such reports include the selected candidate sequence or sequences, a ranking summary, a metric summary, expected advantages, identified tradeoffs, recommended assays, recommended storage conditions, recommended synthesis steps, or combinations thereof. In some embodiments, such reports are used by researchers, designers, synthesis personnel, assay personnel, or regulatory reviewers.

In some embodiments, physical implementation stage 840 and measurement and evaluation stage 850 establish that the disclosed methods are directed to practical, real-world generation and use of biological constructs rather than abstract evaluation alone. In some embodiments, selected sequences are manufactured, tested, stored, expressed, delivered, or otherwise used in a tangible biological context. In some embodiments, such practical use includes biological information storage, target sensing, sequence synthesis, binding optimization, editing workflows, or persistence-oriented sequence deployment.

In some embodiments, the validation and output workflow 800 is configured differently for different application classes. A storage-oriented workflow may emphasize long-term environmental challenge testing, recovery testing, and sequencing fidelity. An aptamer-oriented workflow may emphasize target binding and off-target discrimination. A coding-oriented workflow may emphasize manufacturability, copying fidelity, and expression readouts. An editing-oriented workflow may emphasize on-target editing efficiency, off-target exposure, and template persistence.

In some embodiments, workflow 800 includes one or more stopping rules. In some embodiments, the process terminates after a candidate satisfies one or more required performance thresholds. In some embodiments, the process terminates after a defined number of iterations. In some embodiments, the process terminates when observed improvements fall below a selected threshold or when a required confidence level has been reached.

In some embodiments, workflow 800 includes one or more versioning and traceability features. In some embodiments, each selected candidate is associated with a version identifier, an optimization history, a metric-set version, a weighting profile, an assay profile, and one or more measured-result records. In some embodiments, this facilitates reproducibility, auditability, and downstream decision support.

In some embodiments, workflow 800 includes one or more export pathways for storing selected biological constructs or associated records in one or more databases, digital repositories, laboratory information systems, or storage archives. In some embodiments, this supports long-term reuse, re-analysis, benchmarking, and continued optimization of candidate classes or application-specific design strategies.

The foregoing validation and output embodiments are illustrative and non-limiting. The disclosed invention encompasses any practical workflow in which one or more candidate biological constructs are computationally evaluated using one or more of the disclosed metrics, one or more candidates are selected, and one or more selected candidates are output, generated, synthesized, tested, stored, or otherwise physically implemented.

In some embodiments, the following worked example illustrates an end-to-end implementation of the disclosed optimization workflow, including input definition, candidate generation, metric determination, threshold gating, ranking, and output for physical implementation.

In one worked example, the target biological application comprises DNA-based archival data storage using a library of candidate storage oligonucleotides of length L=150 bases. Input interface 110 receives: (i) a candidate sequence space defined by fixed primer regions and variable payload positions, (ii) an environmental descriptor Env specifying temperature 25° C. and ionic strength 150 mM NaCl, and (iii) an off-target definition comprising other library members intended to coexist in a pooled mixture.

In some embodiments, candidate generation engine 120 generates a candidate set by constrained mutation of the payload positions while enforcing hard constraints including GC content between 40% and 60%, a maximum homopolymer length of 4, and exclusion of one or more forbidden motifs. In one example, 10,000 candidate sequences are generated and stored in a candidate list together with associated descriptor values.

In some embodiments, metric computation engine 130 computes, for each candidate: (a) a structural integrity metric M_struct using a selected structural-profile representation; (b) a melting or separation resistance metric M_melt using at least one environmental descriptor and a calibrated mapping to a persistence-related value; (c) a specificity metric M_spec_multi using an on-target interaction quantity F_on relative to a set of off-target interaction quantities F_off_k; and (d) a fidelity metric M_fidelity using compat_correct and compat_incorrect values computed from a selected mismatch-penalty table, measured error dataset, kinetic model, or calibrated surrogate model.

In some embodiments, scoring and ranking engine 140 applies threshold gating such that a candidate is retained only if M_spec_multi≥T_spec, M_fidelity≥T_fid, and M_struct≤T_struct. In one example, T_spec=10, T_fid=0.5, and T_struct is set proportional to sequence length as T_struct=0.02·L. Remaining candidates are ranked using a composite score with weights selected for a storage-oriented objective in which structural integrity and separation resistance are weighted higher than specificity.

In some embodiments, output interface 150 outputs a ranked list that includes for each selected candidate: the sequence, constraint flags, computed metric values, threshold pass/fail indicators, and a composite score, and generates a synthesis-ready file defining a library of selected sequences for physical synthesis and pooled evaluation. In some embodiments, physical implementation and measurement stages validate retrieval fidelity and cross-hybridization performance, and measured results are recorded and used for calibration in later optimization iterations.

In some embodiments, one or more terms used herein have the following meanings unless the context clearly indicates otherwise. The term biological information-bearing polymer includes DNA, RNA, DNA-RNA hybrids, modified nucleic acids, nucleic acid analogs, and any other sequence-bearing molecular construct capable of storing, transmitting, guiding, encoding, regulating, or otherwise participating in biological information storage or processing.

In some embodiments, the term candidate biological construct refers to any sequence, sequence region, sequence set, sequence library member, vector-associated sequence, template, oligonucleotide, guide, donor, probe, aptamer, regulatory element, coding region, or multi-region construct that is subject to evaluation according to one or more of the disclosed metrics. In some embodiments, a candidate biological construct is fully specified. In some embodiments, a candidate biological construct is partially specified and includes one or more variable positions or variable regions.

In some embodiments, the term sequence descriptor refers to one or more quantities representing nucleotide identity, regional composition, motif content, codon assignments, modification states, methylation states, or derived sequence properties associated with a candidate biological construct. In some embodiments, the term structural descriptor refers to one or more quantities representing conformation, accessibility, geometry, flexibility, curvature, pairing behavior, secondary structure propensity, or other structural features associated with a candidate biological construct. In some embodiments, the term energetic descriptor refers to one or more quantities representing stability, persistence, coherence-related behavior, interaction energies, denaturation resistance, thermal response, or other energy-related properties.

In some embodiments, the term target refers to a desired interaction partner, biological site, molecular species, locus, sequence, motif, environment, or functional outcome for which a candidate biological construct is designed. In some embodiments, the term off-target refers to one or more undesired interaction partners, competing sites, competing molecules, homologous regions, or non-preferred functional outcomes.

In some embodiments, the term recognition science derived refers to a quantity, mapping, parameter, equation, transform, score, or metric that is determined according to one or more principles, constructs, or functions associated with a recognition science framework. In some embodiments, one or more such quantities are used exactly as originally formulated. In other embodiments, one or more such quantities are approximated, transformed, calibrated, discretized, bounded, or otherwise adapted for a selected application while still preserving their intended functional role in the disclosed optimization workflow.

In some embodiments, one or more recognition science derived functions are used in metric determination. In some embodiments, a recognition cost function is represented by:

$$J ( x ) = 0.5 * ( x + 1 / x ) - 1$$

for x greater than 0. In some embodiments, J(x) is used to characterize a cost, overhead, inefficiency, mismatch burden, or related recognition-associated penalty. In some embodiments, J(x) is used directly. In some embodiments, J(x) is used indirectly through one or more derived quantities, normalized forms, approximations, or related transforms.

In some embodiments, a recognition coverage function is represented by:

$$F\_cov ( r ) = r / ( r + X\_rec )$$

where r is a nonnegative recognition-related quantity and X_rec is a recognition constant. In some embodiments, F_cov(r) is used to define one or more overhead, compatibility, fidelity, or selection-related values. In some embodiments, F_cov(r) is applied to measured quantities, simulated quantities, normalized quantities, or theory-derived quantities.

In a preferred embodiment, X_rec is set to phi/pi. In other embodiments, X_rec is assigned according to one or more calibration procedures, target classes, assay contexts, model variants, environmental conditions, sequence classes, or combinations thereof. Accordingly, the disclosed optimization workflow is not limited to a single fixed recognition constant in every embodiment, even where phi/pi is preferred in a recognition science derived implementation.

As used herein, “phi” (also written as “Φ”) denotes the golden ratio, equal to (1+√5)/2, and “pi” (also written as “π”) denotes the circle constant. In some embodiments, the recognition constant X_rec is set to phi/pi, which numerically is approximately 0.515. In other embodiments, X_rec is selected or calibrated as described herein.

In some embodiments, the disclosed framework employs a preferred descriptor representation: D=(S, H, E)where S is a sequence descriptor, H is a structural descriptor, and E is an energetic descriptor. In some embodiments, this representation is useful for compact implementation, theoretical derivation, or baseline optimization. In some embodiments, the representation is broadened to: D=(S, H, E, Env, T, K)where Env represents one or more environmental descriptors, T represents one or more target-related descriptors, and K represents one or more constraints, control parameters, or configuration settings.

In some embodiments, one or more structural descriptors include one or more values associated with pitch, groove ratio, spacing, curvature, flexibility, twist, accessibility, loop behavior, or local geometry. In some embodiments, one or more structural descriptors are represented in a reduced form by a pitch-related parameter P and a groove-related parameter G. In some embodiments, one or more preferred values of P fall within a range from 30 to 40 angstroms. In some embodiments, one or more preferred values of G fall within a range from 1.5 to 1.7. In other embodiments, broader, narrower, overlapping, measured, simulated, or application-specific structural ranges may be used.

In some embodiments, one or more energetic descriptors include a coherence-related energy parameter, a persistence-related parameter, a denaturation-related parameter, an interaction energy parameter, a region-specific energy parameter, or combinations thereof. In some embodiments, one or more energetic descriptors include one or more discrete or quantized levels. In some embodiments, one or more energetic descriptors are determined from one or more measured energy proxies, one or more simulations, one or more calibrated mappings, or combinations thereof.

In some embodiments, sequence descriptors include a stability-related parameter S_stab. In a preferred simple embodiment:

$$S\_stab = 1.5 * ( number of CG base pairs ) + 1. * ( number of AT base pairs )$$

In some embodiments, the same concept is applied with RNA bases, modified bases, hybrid bases, context-specific coefficients, position-specific coefficients, nearest-neighbor coefficients, or learned coefficients. In some embodiments, S_stab is computed globally. In some embodiments, S_stab is computed locally by window, region, or motif.

In some embodiments, the disclosed framework is used with one or more continuous models. In some embodiments, the disclosed framework is used with one or more discrete models. In some embodiments, the disclosed framework is used with one or more lattice-like, graph-based, or region-partitioned models. In some embodiments, the disclosed framework is used with one or more machine-learning-assisted surrogate models. Accordingly, no single mathematical representation is required for every embodiment.

In some embodiments, one or more metric values are determined directly from theory-derived quantities. In some embodiments, one or more metric values are determined from measured biochemical or biophysical values. In some embodiments, one or more metric values are determined from molecular simulation outputs, sequence models, structural predictors, or empirical tables. In some embodiments, one or more metric values are determined from hybrid combinations of theory-derived, measured, and simulated information.

In some embodiments, a hybrid structural metric is represented by:

$$M\_struct \_hybrid = a 0 + a 1 * M\_struct \_RS + a 2 * M\_secondary + a 3 * M\_damage$$

where M_struct_RS is a recognition-science-derived structural metric, M_secondary is a secondary-structure-related quantity, M_damage is a damage-related quantity, and a0, a1, a2, and a3 are coefficients. In some embodiments, the coefficients are fixed. In some embodiments, the coefficients are calibrated. In some embodiments, the coefficients are learned or adaptively updated.

In some embodiments, a hybrid specificity metric is represented by:

$$M\_spec \_hybrid = b 0 + b 1 * M\_spec \_RS + b 2 * M\_access + b 3 * M\_offpen$$

where M_spec_RS is a recognition-science-derived specificity metric, M_access is an accessibility-related quantity, M_offpen is an off-target-penalty-related quantity, and b0, b1, b2, and b3 are coefficients. In some embodiments, one or more additional terms are included or substituted.

In some embodiments, a hybrid fidelity metric is represented by:

$$M\_fidelity \_hybrid = c 0 + c 1 * M\_fidelity \_RS + c 2 * M\_poly + c 3 * M\_errorhist$$

where M_fidelity_RS is a recognition-science-derived fidelity metric, M_poly is a polymerase- or processing-related quantity, M_errorhist is an observed or inferred error-history quantity, and c0, c1, c2, and c3 are coefficients. In some embodiments, such a hybrid metric improves application-specific fit while preserving the general recognition-based structure of the optimization framework.

In some embodiments, one or more metrics are determined at the level of the entire construct. In some embodiments, one or more metrics are determined for one or more local regions. In some embodiments, local region metrics are aggregated according to one or more region weights, region priorities, region classes, or region-specific selection rules. In some embodiments, a first region may be treated as a critical motif region, a second region as a spacer region, a third region as a coding region, and a fourth region as a handling or amplification region.

In some embodiments, a region-aware score is represented by:

$$Score\_multi = sum over regions j of q\_j * Score\_j$$

where Score_j is a region-level score and q_j is a region-level weighting factor. In some embodiments, q_j is selected according to region function, sequence length, target criticality, position, assay sensitivity, or user preference.

In some embodiments, one or more candidate generation workflows are tailored to region-aware optimization. In some embodiments, one or more motif positions are held fixed while flanking positions are varied. In some embodiments, one or more coding positions are synonymously varied while noncoding positions are freely varied within one or more constraint sets. In some embodiments, one or more primer regions are constrained to preserve amplification behavior while storage regions or payload regions are optimized for persistence and separability.

In some embodiments, the disclosed optimization framework is used for DNA storage embodiments. In some embodiments, a storage construct includes one or more payload regions, index regions, primer regions, parity regions, synchronization regions, control regions, barcodes, or combinations thereof. In some embodiments, storage sequences are selected to maximize persistence, minimize cross-hybridization, improve amplification robustness, and maintain compatibility with sequencing or retrieval workflows.

In some embodiments, the disclosed optimization framework is used for aptamer, probe, or recognition-sequence embodiments. In some embodiments, a target panel and one or more off-target panels are explicitly provided. In some embodiments, one or more local structures, loops, or recognition footprints are preserved or encouraged. In some embodiments, selected candidates are output as ranked panels for subsequent laboratory screening rather than as a single final sequence.

In some embodiments, the disclosed optimization framework is used for coding-sequence embodiments. In some embodiments, the encoded amino acid sequence is preserved as a hard constraint while one or more synonymous codon choices are varied. In some embodiments, local structure, fidelity, synthesis feasibility, expression-related behavior, or combinations thereof are optimized. In some embodiments, one or more folding-related, trajectory-related, or downstream-realization-related quantities are used as supplemental metrics.

In some embodiments, the disclosed optimization framework is used for regulatory-sequence embodiments. In some embodiments, a candidate construct includes one or more promoter segments, enhancer segments, operator segments, transcription-factor-binding footprints, untranslated regions, splice-related motifs, or combinations thereof. In some embodiments, target specificity and local accessibility are jointly optimized. In some embodiments, flanking regions are varied while one or more core motifs are preserved.

In some embodiments, the disclosed optimization framework is used for guide-sequence, donor-template, repair-template, or genome-editing-related embodiments. In some embodiments, one or more target loci and one or more off-target loci are identified and used in specificity analysis. In some embodiments, fidelity and compatibility metrics are used to favor candidates expected to improve desired editing outcomes or reduce undesired processing events. In some embodiments, donor-template persistence and guide stability are also optimized.

In some embodiments, the disclosed optimization framework is used for RNA-related embodiments. In some embodiments, such embodiments include mRNA design, antisense design, siRNA design, ribozyme design, guide RNA design, and RNA aptamer design. In some embodiments, RNA-specific structural forms, accessibility properties, modification states, processing properties, or stability factors are included in the descriptors or metrics.

In some embodiments, the disclosed optimization framework is used for modified-nucleic-acid embodiments. In some embodiments, sequence descriptors or energetic descriptors are augmented to account for methylation, hydroxymethylation, base analogs, backbone modifications, sugar modifications, or combinations thereof. In some embodiments, one or more metric mappings are adjusted to account for modified-nucleic-acid persistence, recognition, or processing behavior.

In some embodiments, one or more environmental descriptors are explicitly incorporated into candidate generation or candidate scoring. In some embodiments, such environmental descriptors include temperature, pH, ionic strength, solvent condition, hydration state, crowding condition, storage condition, cellular condition, assay condition, or combinations thereof. In some embodiments, a candidate is selected not for a single universal environment but for a defined intended-use environment.

In some embodiments, one or more outputs include a single selected sequence. In some embodiments, one or more outputs include a ranked list, a threshold-qualified set, a Pareto front, a diversity-preserved panel, a sequence library, a construct record, a synthesis instruction set, or an assay plan. In some embodiments, one or more outputs are directly consumed by laboratory workflows, storage systems, manufacturing systems, or downstream computational tools.

In some embodiments, one or more optimized sequences or constructs produced according to the disclosed method are physically synthesized, cloned, amplified, transcribed, packaged, immobilized, stored, assayed, delivered, or otherwise implemented in a tangible context. In some embodiments, this includes use in storage media, biosensors, diagnostic assays, guide systems, donor systems, expression constructs, sequence libraries, or related biological systems.

In some embodiments, the disclosed methods are implemented by one or more processors executing instructions stored in one or more non-transitory computer-readable media. In some embodiments, the disclosed methods are implemented by one or more distributed systems, cloud systems, local systems, accelerators, or combinations thereof. In some embodiments, the disclosed methods are embodied in software, firmware, hardware, or combinations thereof.

In some embodiments, the disclosed methods employ exact formulas. In some embodiments, the disclosed methods employ approximate formulas. In some embodiments, the disclosed methods employ discrete analogs, bounded analogs, monotone-equivalent analogs, normalized analogs, or transformed analogs. In some embodiments, two formulations are treated as functionally equivalent where they preserve the intended ranking, selection, or optimization relationship among candidates.

In some embodiments, one or more search strategies are deterministic. In some embodiments, one or more search strategies are stochastic. In some embodiments, one or more search strategies are evolutionary, heuristic, or machine-learning-guided. In some embodiments, the selected search strategy depends on sequence length, sequence-space size, target complexity, computational budget, required throughput, or intended application.

In some embodiments, one or more candidate selections are based on scalar composite scoring. In some embodiments, one or more candidate selections are based on Pareto selection. In some embodiments, one or more candidate selections are based on thresholding. In some embodiments, one or more candidate selections are based on combinations of thresholding, scalar scoring, Pareto logic, diversity preservation, uncertainty management, or iterative validation feedback.

In some embodiments, one or more parameters, coefficients, transformations, or thresholds are fixed in advance. In some embodiments, one or more parameters, coefficients, transformations, or thresholds are selected by a user. In some embodiments, one or more parameters, coefficients, transformations, or thresholds are learned, calibrated, inferred, adaptively updated, or retrieved from one or more prior runs or reference datasets.

Although particular embodiments have been described herein with reference to DNA, the disclosed framework is not limited to DNA unless expressly stated. The same or analogous framework may be applied to RNA, DNA-RNA hybrids, modified nucleic acids, and other biological information-bearing polymers where compatible with the intended application.

Although particular embodiments have been described herein with reference to selected applications, including storage, aptamer design, biosensor design, coding optimization, regulatory-sequence optimization, and editing-related design, the disclosed framework is not limited to such examples. The disclosed framework may be applied to any biological information storage or processing problem in which one or more candidate constructs are evaluated according to one or more structural, energetic, specificity-related, fidelity-related, or recognition-related criteria.

Features described in connection with one embodiment may be combined with features described in connection with another embodiment unless incompatible. Separate modules, stages, engines, or workflows described herein may be combined, subdivided, reordered, or omitted in selected implementations. Likewise, steps described as occurring in one order may occur in another order unless a particular order is expressly required.

Terms such as may, can, in some embodiments, in preferred embodiments, optionally, and the like are intended to reflect non-limiting examples rather than mandatory limitations. Numerical ranges described herein include subranges and individual values within the stated ranges. Mathematical expressions described herein include exact forms, approximations, transformed forms, normalized forms, bounded forms, discretized forms, and monotone-equivalent forms where appropriate to the described function.

The foregoing description of embodiments is illustrative and not limiting. The invention is defined by the claims, and all changes, substitutions, modifications, and equivalents that fall within the spirit and scope of the claims are intended to be embraced therein.

What is claimed is:

1. A computer-implemented method for designing a biological information-bearing polymer, the method comprising:
receiving, by one or more processors, input data defining (i) a target biological application and (ii) one or more candidate biological information-bearing polymers or a candidate sequence space;
generating, by the one or more processors, a candidate set of biological information-bearing polymers from the one or more candidate biological information-bearing polymers or the candidate sequence space, including applying one or more constraints to generate or filter the candidate set;
determining, by the one or more processors, for at least one candidate biological information-bearing polymer of the candidate set, a structural integrity metric, a melting or separation resistance metric, a binding specificity metric based at least in part on at least one on-target interaction quantity and one or more off-target interaction quantities, and a replication or transcription fidelity metric based at least in part on a correct-event compatibility quantity and an incorrect-event compatibility quantity;
determining, by the one or more processors, a selection result by applying one or more threshold conditions to at least the binding specificity metric, the replication or transcription fidelity metric, and the structural integrity metric, and ranking candidates that satisfy the one or more threshold conditions using a composite score based at least in part on the structural integrity metric, the melting or separation resistance metric, the binding specificity metric, and the replication or transcription fidelity metric; and outputting, by the one or more processors, one or more selected biological information-bearing polymers as a ranked candidate list, a sequence library, or a synthesis-ready construct definition for physical synthesis, assembly, storage, screening, or use.

2. The method of claim 1, wherein the one or more candidate biological information-bearing polymers comprise DNA, RNA, a DNA-RNA hybrid, a modified nucleic acid, or a combination thereof.

3. The method of claim 1, wherein determining the plurality of metrics comprises representing the at least one candidate biological information-bearing polymer using at least a sequence descriptor, a structural descriptor, and an energetic descriptor.

4. The method of claim 1, wherein the structural integrity metric is based at least in part on deviation of a structural profile from a reference structural profile over a length, region, or coordinate domain of the at least one candidate biological information-bearing polymer.

5. The method of claim 1, wherein the melting or separation resistance metric is based at least in part on a stability-related quantity and an energy-related quantity associated with denaturation resistance, dissociation resistance, or strand-separation resistance.

6. The method of claim 1, wherein the on-target interaction quantity and the one or more off-target interaction quantities are computed from at least one of predicted binding free energy, predicted affinity or occupancy, a motif or sequence-model score, simulated interaction outputs, or experimentally measured binding, cleavage, enrichment, or reporter assay data, including applying a common computation pathway or normalization to the on-target interaction quantity and the one or more off-target interaction quantities.

7. The method of claim 6, wherein the binding specificity metric is computed based at least in part on an on-target interaction amplitude and a plurality of off-target interaction amplitudes according to:
$$M\_spec \_multi = ( abs ( F\_on ) \hat{} 2 ) / ( sum over k of abs ( F\_off \_k ) \hat{} 2 ) ,$$
where k indexes the plurality of off-target interactions.

8. The method of claim 1, wherein the correct-event compatibility quantity and the incorrect-event compatibility quantity are nonnegative quantities on a common scale, and are determined from at least one of a mismatch-penalty table, a polymerase-or ligase-specific kinetic model, empirically measured misincorporation or error-spectrum data, sequencing-derived error statistics, assay signal data mapped into compatibility values, simulated outcomes, or a calibrated surrogate model trained on measured datasets.

9. The method of claim 8, wherein the replication or transcription fidelity metric is computed according to:
$$M\_fidelity = X\_rec * ( 1 / compat\_incorrect - 1 / compat\_correct ) ,$$
where X_rec is a positive recognition constant.

10. The method of claim 1, wherein determining one or more of the structural integrity metric, the binding specificity metric, or the replication or transcription fidelity metric comprises applying a calibrated mapping that uses a recognition constant X_rec, and wherein X_rec is selected or fit based at least in part on the target biological application, an environmental descriptor, or measured assay data associated with one or more reference polymers.

11. The method of claim 1, wherein determining the selection result comprises applying one or more normalization operations, weighting operations, threshold conditions, or Pareto selection criteria to the plurality of metrics, wherein:
the weighting operations apply weights w1 to w4 to respective ones of the structural integrity metric, the melting or separation resistance metric, the binding specificity metric, and the replication or transcription fidelity metric; and
at least one normalization operation maps the structural integrity metric using 1/(M_struct+epsilon), where epsilon is a nonzero stabilizing quantity, and optionally maps the replication or transcription fidelity metric using M_fidelity/(M_fidelity+k_f), where k_f is a positive scale-setting quantity.

12. The method of claim 11, wherein the threshold conditions comprise retaining a candidate only if:
(i) M_spec_multi≥T_spec,
(ii) M_fidelity≥T_fid, and
(iii) M_struct≤T_struct,
where T_spec, T_fid, and T_struct are threshold parameters, and wherein at least one of (a) T_struct is set proportional to a sequence length L of the candidate, (b) T_spec is in a range from 5 to 50, or (c) T_fid is in a range from 0.1 to 5.

13. The method of claim 1, further comprising generating the one or more candidate biological information-bearing polymers by constrained enumeration, mutation, substitution, codon optimization, motif-preserving editing, stochastic search, evolutionary search, machine-learning-guided search, or a combination thereof.

14. The method of claim 1, further comprising applying one or more hard constraints or soft constraints to candidate generation, candidate filtering, candidate scoring, or candidate selection, wherein the one or more hard constraints or soft constraints comprise sequence length constraints, motif constraints, homopolymer constraints, codon-preservation constraints, amino-acid-preservation constraints, or synthesis constraints.

15. The method of claim 1, wherein outputting the one or more selected biological information-bearing polymers comprises outputting a ranked candidate list, a sequence library, a synthesis-ready construct definition, a manufacturing instruction set, an assay plan, or a database record, and further comprising causing physical implementation of at least one selected biological information-bearing polymer by synthesizing, cloning, amplifying, transcribing, packaging, immobilizing, storing, screening, or delivering the at least one selected biological information-bearing polymer.

16. A system for designing a biological information-bearing polymer, the system comprising:
one or more processors; and
one or more non-transitory computer-readable media storing instructions that, when executed by the one or more processors, cause the system to:
receive input data defining (i) a target biological application and (ii) one or more candidate biological information-bearing polymers or a candidate sequence space;
generate a candidate set from the one or more candidate biological information-bearing polymers or the candidate sequence space, including applying one or more constraints to generate or filter the candidate set;
determine, for at least one candidate of the candidate set, a structural integrity metric, a melting or separation resistance metric, a binding specificity metric based at least in part on at least one on-target interaction quantity and one or more off-target interaction quantities, and a replication or transcription fidelity metric based at least in part on a correct-event compatibility quantity and an incorrect-event compatibility quantity;
determine a selection result by applying one or more threshold conditions to at least the binding specificity metric, the replication or transcription fidelity metric, and the structural integrity metric, and ranking candidates that satisfy the one or more threshold conditions using a composite score based at least in part on the structural integrity metric, the melting or separation resistance metric, the binding specificity metric, and the replication or transcription fidelity metric; and
output one or more selected biological information-bearing polymers as a ranked candidate list, a sequence library, or a synthesis-ready construct definition for physical synthesis, assembly, storage, screening, or use.

17. The system of claim 16, wherein the instructions further cause the system to generate candidate biological information-bearing polymers, apply one or more constraints, rank a plurality of candidate biological information-bearing polymers, and output a ranked candidate list or a synthesis-ready construct definition.

18. The system of claim 16, wherein the instructions further cause the system to update one or more parameters, thresholds, weights, or metric mappings based at least in part on measured assay data associated with one or more previously selected biological information-bearing polymers.

19. A non-transitory computer-readable medium storing instructions that, when executed by one or more processors, cause the one or more processors to:
receive input data defining (i) a target biological application and (ii) one or more candidate biological information-bearing polymers or a candidate sequence space;
generate a candidate set from the one or more candidate biological information-bearing polymers or the candidate sequence space, including applying one or more constraints to generate or filter the candidate set;
determine, for at least one candidate of the candidate set, a structural integrity metric, a melting or separation resistance metric, a binding specificity metric based at least in part on at least one on-target interaction quantity and one or more off-target interaction quantities, and a replication or transcription fidelity metric based at least in part on a correct-event compatibility quantity and an incorrect-event compatibility quantity;
determine a selection result by applying one or more threshold conditions to at least the binding specificity metric, the replication or transcription fidelity metric, and the structural integrity metric, and ranking candidates that satisfy the one or more threshold conditions using a composite score based at least in part on the structural integrity metric, the melting or separation resistance metric, the binding specificity metric, and the replication or transcription fidelity metric; and
output one or more selected biological information-bearing polymers as a ranked candidate list, a sequence library, or a synthesis-ready construct definition for physical synthesis, assembly, storage, screening, or use.

20. The non-transitory computer-readable medium of claim 19, wherein the instructions further cause the one or more processors to output a sequence library, a synthesis-ready construct definition, or an assay plan for laboratory validation.
